(19) United States
(12) Patent Application Publication
(10) Pub. No.: US 2002/0065219 A1
(43) Pub. Date: May 30, 2002

(54) Water soluble thiazolyl peptide derivatives

(76) Inventors: B. Narasimhulu Naidu, Durham, CT (US); Wenying Li, Middletown, CT (US); Kin S. Lam, North Haven, CT (US); Margaret E. Sorenson, Meriden, CT (US); John A. Wichtowski, Deep River, CT (US); Timothy P. Connolly, Meriden, CT (US); Yasutsugu Ueda, Clinton, CT (US); Joanne J. Bronson, Durham, CT (US); Yunhui Zhang, Glastonbury, CT (US); Oak K. Kim, Milford, CT (US)

Correspondence Address: Marla J. Mathias, Bristol-Myers Squibb Company, Patent Department, P.O. Box 4000, Princeton, NJ, 08543-4000, US

(21) Appl. No.: 09/928,468

(22) Filed: Aug. 13, 2001

Related U.S. Application Data
(60) Provisional application No. 60/225,598, filed on Aug. 15, 2000.

(51) Int. Cl. A61K 38/12; C07K 7/50

(52) U.S. Cl. 514/9; 530/317; 540/456

(57) ABSTRACT

Novel thiazolyl peptide compounds useful for the treatment of serious bacterial infections, and pharmaceutical compositions thereof are provided. More particularly, novel thiazolyl peptide compounds are provided having the general formula I:

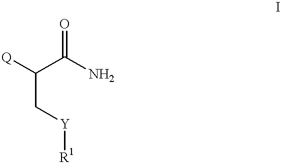

wherein Q, Y and R′ areas defined in the specification.

### CROSS REFERENCE TO RELATED APPLICATION

This application claims the benefit of U.S. Provisional Application Serial No. 60/225,598 filed on Aug. 15, 2000.

### FIELD OF THE INVENTION

This invention relates to novel thiazolyl peptide compounds useful for the treatment of serious bacterial infections and suitable for both oral and, particularly, parenteral administration. This invention also relates to a pharmaceutical composition, especially an antibacterial composition, which comprises the novel thiazolyl peptide derivative as an active ingredient. The invention also provides a method for treating serious bacterial infections by administering to a mammal in need thereof said thiazolyl peptide compounds or a pharmaceutical composition of the thiazolyl peptide compounds.

### BACKGROUND OF THE INVENTION

Multidrug-resistant strains of many clinically important pathogenic bacteria, including methicillin-resistant Staphylococcus aureus (MRSA), Streptococcus pneumoniae, Mycobacterium tuberculosis, and Enterococci strains are becoming a worldwide health problem. There is an urgent need to discover new agents to treat patients infected with multidrug-resistant bacteria. Many thiazolyl peptide antibiotics possess potent antimicrobial activity against Gram-positive bacteria, including multidrug-resistant strains, however many of these compounds have poor water solubility. The poor water solubility possessed by many of the thiazolyl peptide antibiotics poses a number of serious limitations to their method of administration and use as therapeutic agents. It is therefore desirable to have thiazolyl peptide antibiotics which are water soluble and can be readily administered while maintaining potent antimicrobial activity. Novel thiazolyl peptide antibiotics, having inhibitory activity at the nanomolar level against Gram-positive bacteria, have been discovered. The thiazolyl peptide compounds described herein exhibit potent antimicrobial activity against Gram-positive bacteria in vitro, and exhibit in vivo efficacy in a systemic Staph. aureus infection model in animals. Many of the compounds of the present invention are water-soluble antibiotics, generally with a solubility of 2-10 mg/mL in water at a pH of 2-4. Many of the compounds are suitable for parenteral use in the treatment of serious bacterial infections in mammals, and more particularly, in humans.

### BACKGROUND OF THE INVENTION

The novel thiazolyl peptide antibiotics of this invention are derived from thiazolyl peptide antibiotics such as the nocathiacins described by J. E. Leet et al in U.S. Pat. No. 6,218,398 (issued Apr. 17, 2001), PCT Appl. WO 00/03722 (published Jan. 27, 2000); PCT Appl. WO 00/14100 and Sasaki, T. et al., J. of Antibiotics 51, No. 8, pp. 715-721 (1998); and nosiheptide (Prange, T. et al., J. Am Chem Soc. 1977, 99, 6418; Benazet, F. et al., Experientia 1980, 36, 414; Floss, H. G. et al., J. Am Chem Soc.1993, 115, 7557). Other novel water-soluble thiazolyl peptide antibiotics may be derived from thiazolyl peptide antibiotics such as Antibiotic S-54832A (Keller-Juslen, C. et al., U.S. Pat. No. 4,478,831, 1984); thiostrepton (Anderson, B. et al., Nature 1970, 225, 233-235; U.S. Pat. No. 2,982,689, 1961 and U.S. Pat. No. 2,982,698, 1961); thiopeptin; methylsulfomycin I (Kumar, V.; Kenia, J.; Mukhopadhyay, T.; Nadkarni, S. R. J. Nat Prod. 1999, 62,1562-1564; GE37468 (Stella, S. et al J. of Antibiotics 1995, 48(8), 780-786); Sch 40832 (Puar, M. S. et al. in J. of Antibiotics 1998, 51(2), 221-224); promothiocins (Yun, B. S. et al. J. Antibiotics 1994, 47, 510-514 and Bagley, M. C. et al. J. Am. Chem. Soc. 2000, 122, 3301-3313) according to the methods described herein.

### SUMMARY OF THE INVENTION

This invention relates to novel thiazolyl peptide compounds, pharmaceutical compositions which contain the novel thiazolyl peptide compounds, and methods of treating mammals which have or are susceptible to serious bacterial infections.

Accordingly, compounds of Formula I, including pharmaceutically acceptable salts thereof, have been discovered which possess potent antibiotic activity, particularly in inhibiting the growth of Gram-positive bacteria and mycobacteria. The compounds are of Formula I

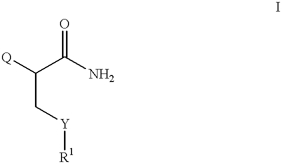

wherein:

Q is a residue of a thiazolyl peptide antibiotic selected from:

[chemical structure]

Y is NR or S(O)ₘ;

m is 0, 1, or 2;

W is selected from the group consisting of hydrogen,

[chemical structure]

R is selected from the group consisting of hydrogen, hydroxy, C₁₋₆alkoxy, —[(CH₂)₂O]ₚ(CH₂)₂R⁴₁, —C(O)C₁₋₆alkyl, —C(O)C₁₋₆alkylCO₂H, —C(O)NHC₁₋₆alkyl and C₁₋₈alkyl, in which said C₁₋₈alkyl is optionally substituted by one to six hydroxy and optionally substituted by one to two same or different substituents selected from the group consisting of (a)-(h):

(a) CO₂R⁵;

(b) SO₃H;

(c) NR⁶R⁷;

(d) heteroaryl, in which said heteroaryl is selected from the group consisting of pyridinyl, pyrimidinyl, pyrazolyl, pyrrolyl, imidazolyl, triazolyl and tetrazolyl, and in which said heteroaryl is optionally substituted with one or two same or different nitro or C₁₋₄alkyl;

(e) phenyl, in which said phenyl is optionally substituted with one to three C₁₋₄alkoxy or optionally substituted with one

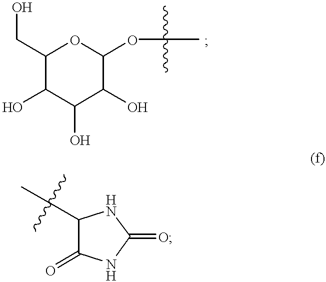

(g) C₁₋₄alkoxy; and

(h) —C(O)NH-heteroaryl, in which said heteroaryl is selected from the group consisting of pyridinyl, pyrimidinyl, pyrazolyl, pyrrolyl, imidazolyl, triazolyl and tetrazolyl;

R′ is selected from the group consisting of:

[chemical structure]

, hydrogen, —[(CH₂)₂O]_{p′}(CH₂)₂R^{4′} and C₁₋₈alkyl, in which said C₁₋₈alkyl is optionally substituted by one to six hydroxy and optionally substituted by one to two same or different substituents selected from the group consisting of (a)-(h):

(a) CO₂R^{5′};

(b) SO₃H;

(c) NR^{6′}R^{7′}

(d) heteroaryl, in which said heteroaryl is selected from the group consisting of pyridinyl, pyrimidinyl, pyrazolyl, pyrrolyl, imidazolyl, triazolyl and tetrazolyl, and in which said heteroaryl is optionally substituted with one or two same or different nitro or C₁₋₄alkyl;

(e) phenyl, in which said phenyl is optionally substituted with one to three C₁₋₄alkoxy or optionally substituted with one

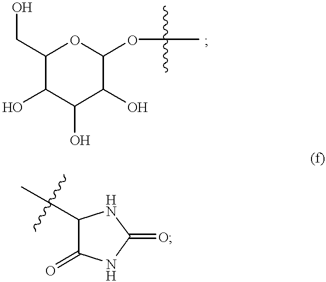

(g) C₁₋₄alkoxy; and

(h) —C(O)NH-heteroaryl, in which said heteroaryl is selected from the group consisting of pyridinyl, pyrimidinyl, pyrazolyl, pyrrolyl, imidazolyl, triazolyl and tetrazolyl;

or R and R¹ together with the nitrogen to which they are attached form a heteroalicyclic selected from the group consisting of:

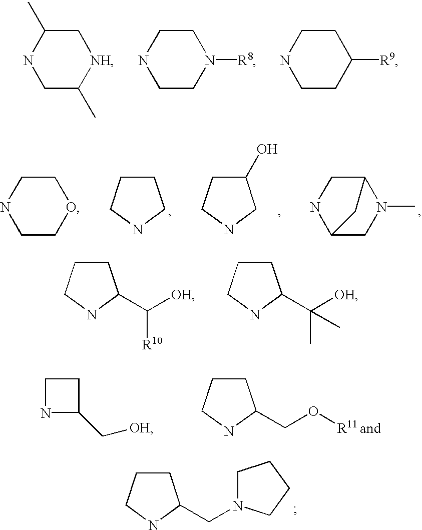

R² is selected from the group consisting of hydrogen, hydroxy, —OC(O) C₁₋₆alkyl and —OC(O)NHC₁₋₆alkyl;

R³ is hydrogen or

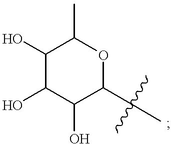

p and p′ are each independently selected from the group consisting of 1, 2 and 3;

R⁴ and R^{4′} are each independently selected from the group consisting of hydroxy, amino and C₁₋₄alkoxy;

R⁵ and R^{5═} are each independently selected from the group consisting of hydrogen, C₁₋₆alkyl and phenylmethyl;

R⁶, R^{6′}, R⁷ and R^{7′} are each independently selected from the group consisting of hydrogen, —C(O)C₁₋₆alkyl, pyridinyl and C₁₋₆alkyl, in which said C₁₋₆alkyl is optionally substituted with one hydroxy, amino, C₁₋₄alkylamino, or di(C₁₋₄alkyl)amino,

or R⁶ and R⁷ taken together with the nitrogen to which they are attached, or R^{6′} and R^{7′} taken together with the nitrogen to which they are attached form a heteroalicyclic selected from the group consisting of succinimid-1-yl, pyrrolidin-2-one-1-yl, pyrrolidin-1-yl, piperidin-1-yl, 4-hydroxypiperidin-1-yl, morpholin-4-yl, piperazin-1-yl and 4-methylpiperazin-1-yl;

R⁸ is selected from the group consisting of C₁₋₆alkyl, —C(O)C₁₋₆alkyl, —[(CH₂)₂O]_{q}(CH₂)₂R^{8′}, pyridinyl and pyrimidinyl, in which said C₁₋₆alkyl is optionally substituted with one di(C₁₋₄alkyl)amino, morpholin-4-yl, CO₂H, —CO₂C₁₋₄alkyl, tri(C₁₋₄alkoxy)phenyl and di(C₁₋₄alkoxy)pyrimidinyl;

q is 1, 2 or 3;

R^{8′} is selected from the group consisting of hydroxy, amino and C₁₋₄alkoxy;

R⁹ is hydrogen or hydroxy;

R¹⁰ is selected from the group consisting of hydrogen, C₁₋₆alkyl, C₃₋₆cycloalkyl and 1-methyl-1H-imidazol-2-yl; and

R¹¹ is C₁₋₄alkyl or pyridinyl.

Pharmaceutical compositions containing a compound of Formula I in addition to a pharmaceutically acceptable carrier, adjuvant or diluent are within the scope of this invention. Methods of treating or preventing bacterial or mycobacterial infections in a mammal in need thereof by administering a compound of Formula I to said mammal are also within the scope of the present invention.

### DETAILED DESCRIPTION OF THE INVENTION

The description of the invention herein should be construed in congruity with the laws and principals of chemical bonding. An embodiment or aspect which depends from another embodiment or aspect, will describe only the variables having values and provisos that differ from the embodiment or aspect from which it depends. Thus, for example, an embodiment which reads “the compound of formula I according to the n^{th} aspect of the invention, wherein R¹ is C₁₋₈alkyl should be read to include all remaining variables with values defined in the n^{th} aspect and should be read to further include all the provisos, unless otherwise indicated, pertaining to each and every variable in the n^{th} aspect.

A first embodiment of a first aspect of the present invention is a compound of Formula I, including pharmaceutically acceptable salts thereof,

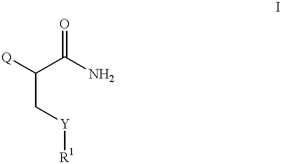

wherein:

Q is a residue of a thiazolyl peptide antibiotic selected from:

[chemical structure]

Y is NR or S(O)ₘ;

m is 0, 1, or 2;

W is selected from the group consisting of hydrogen,

[chemical structure]

R is selected from the group consisting of hydrogen, hydroxy, C₁₋₆alkoxy, —[(CH₂)₂O]ₚ(CH₂)₂R⁴, —C(O)C₁₋₆alkyl, —C(O)C₁₋₆alkylCO₂H, —C(O)NHC₁₋₆alkyl and C₁₋₈alkyl, in which said C₁₋₈alkyl is optionally substituted by one to six hydroxy and optionally substituted by one to two same or different substituents selected from the group consisting of (a)-(h):

(a) CO₂R⁵;

(b) SO₃H;

(c) NR⁶R⁷;

(d) heteroaryl, in which said heteroaryl is selected from the group consisting of pyridinyl, pyrimidinyl, pyrazolyl, pyrazinyl, pyrrolyl, imidazolyl, triazolyl and tetrazolyl, and in which said heteroaryl is optionally substituted with one or two same or different nitro or C₁₋₄alkyl;

(e) phenyl, in which said phenyl is optionally substituted with one to three C₁₋₄alkoxy or optionally substituted with one

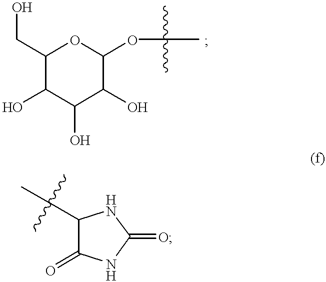

(g) C₁₋₄alkoxy; and

(h) —C(O)NH-heteroaryl, in which said heteroaryl is selected from the group consisting of pyridinyl, pyrimidinyl, pyrazolyl, pyrrolyl, imidazolyl, triazolyl and tetrazolyl;

R¹ is selected from the group consisting of:

[chemical structure]

, hydrogen, —[(CH₂)₂O]_{p′}(CH₂)₂R^{4′} and C₁₋₈alkyl, in which said C₁₋₈alkyl is optionally substituted by one to six hydroxy and optionally substituted by one to two same or different substituents selected from the group consisting of (a)-(h):

(a) CO₂R^{5′};

(b) SO₃H;

(c) NR^{6′}R^{7′};

(d) heteroaryl, in which said heteroaryl is selected from the group consisting of pyridinyl, pyrimidinyl, pyrazolyl, pyrazinyl, pyrrolyl, imidazolyl, triazolyl and tetrazolyl, and in which said heteroaryl is optionally substituted with one or two same or different nitro or C₁₋₄alkyl;

(e) phenyl, in which said phenyl is optionally substituted with one to three C₁₋₄alkoxy or optionally substituted with one

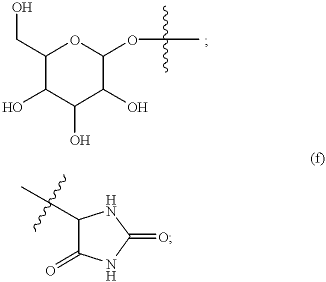

(g) C₁₋₄alkoxy; and

(h) —C(O)NH-heteroaryl, in which said heteroaryl is selected from the group consisting of pyridinyl, pyrimidinyl, pyrazolyl, pyrrolyl, imidazolyl, triazolyl and tetrazolyl;

or R and R¹ together with the nitrogen to which they are attached form a heteroalicyclic selected from the group consisting of:

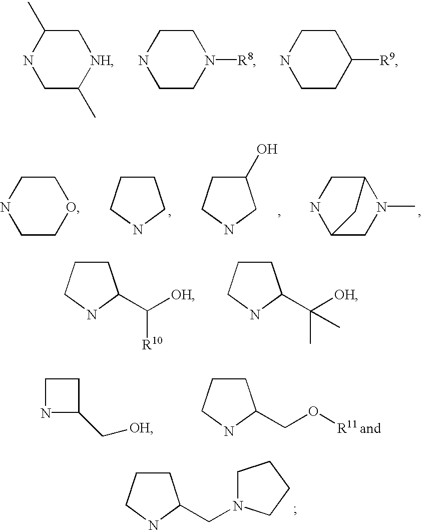

R² is selected from the group consisting of hydrogen, hydroxy, —OC(O) C₁₋₆alkyl and —OC(O)NHC₁₋₆alkyl;

R³ is hydrogen or

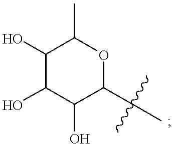

p and p′ are each independently selected from the group consisting of 1, 2 and 3;

R⁴ and R^{4′} are each independently selected from the group consisting of hydroxy, amino and C₁₋₄alkoxy;

R⁵ and R^{5′} are each independently selected from the group consisting of hydrogen, C₁₋₆alkyl and phenylmethyl;

R⁶, R^{6′}, R⁷ and R^{7′} are each independently selected from the group consisting of hydrogen, —C(O)C₁₋₆alkyl, pyridinyl and C₁₋₆alkyl, in which said C₁₋₆alkyl is optionally substituted with one hydroxy, amino, C₁₋₄alkylamino, or di(C₁₋₄alkyl)amino,

or R⁶ and R⁷ taken together with the nitrogen to which they are attached, or R^{6′} and R^{7′} taken together with the nitrogen to which they are attached form a heteroalicyclic selected from the group consisting of succinimid-1-yl, pyrrolidin-2-one-1-yl, pyrrolidin-1-yl, piperidin-1-yl, 4-hydroxypiperidin-1-yl, morpholin-4-yl, piperazin-1-yl and 4-methylpiperazin-1-yl;

R⁸ is selected from the group consisting of C₁₋₆alkyl, —C(O)C₁₋₆alkyl, —[(CH₂)₂O]_{q}(CH₂)₂R^{8′}, pyridinyl and pyrimidinyl, in which said C₁₋₆alkyl is optionally substituted with one di(C₁₋₄alkyl)amino, morpholin-4-yl, CO₂H, —CO₂C₁₋₄alkyl, tri(C₁₋₄alkoxy)phenyl and di(C₁₋₄alkoxy)pyrimidinyl;

q is 1, 2 or 3;

R^{8′} is selected from the group consisting of hydroxy, amino and C₁₋₄alkoxy;

R⁹ is hydrogen or hydroxy;

R¹⁰ is selected from the group consisting of hydrogen, C₁₋₆alkyl, C₃₋₆cycloalkyl and 1-methyl-1H-imidazol-2-yl; and

R¹¹ is C₁₋₄alkyl or pyridinyl.

A second embodiment of a first aspect of the present invention is compound of the first embodiment, including pharmaceutically acceptable salts thereof, wherein Q is

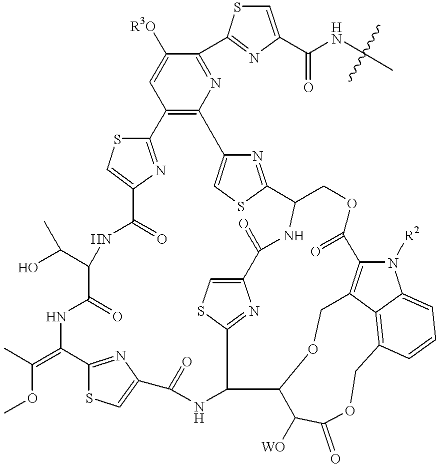

A third embodiment of a first aspect of the present invention is a compound of the second embodiment, including pharmaceutically acceptable salts thereof, wherein W is selected from the group consisting of hydrogen,

[chemical structure]

A fourth embodiment of a first aspect of the present invention is a compound of the third embodiment, including pharmaceutically acceptable salts thereof, wherein Y is NR.

A fourth embodiment of a first aspect of the present invention is a compound of the fourth embodiment, including pharmaceutically acceptable salts thereof, wherein R is selected from the group consisting of hydrogen, hydroxy, —C(O)CH₃, —C(O)(CH₂)₂CO₂H, —C(O)NHCH₃ and C₁₋₄alkyl, in which said C₁₋₄alkyl is optionally substituted with one hydroxy or di(C₁₋₄alkyl)amino.

A sixth embodiment of a first aspect of the present invention is a compound of the fourth embodiment, including pharmaceutically acceptable salts thereof, wherein NRR¹ is selected from the group consisting of:

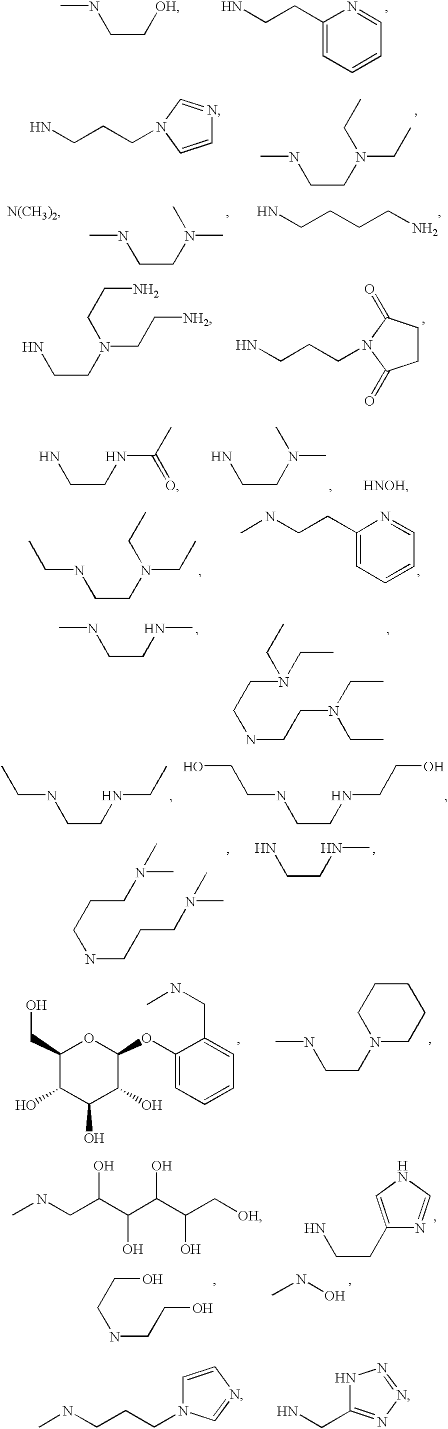

A seventh embodiment of a first aspect of the present invention is a compound of the sixth embodiment, including pharmaceutically acceptable salts thereof, wherein W is

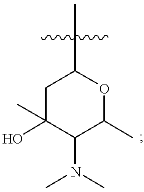

R² is hydroxy; and R³ is hydrogen.

An eighth embodiment of the present invention is a compound of the sixth embodiment, including pharmaceutically acceptable salts thereof, wherein R² and R³ are each hydrogen.

A ninth embodiment of a first aspect of the present invention is a compound of the eighth embodiment, including pharmaceutically acceptable salts thereof, wherein W is

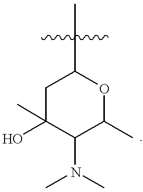

A tenth embodiment of a first aspect of the present invention is the compound of the ninth embodiment, including pharmaceutically acceptable salts thereof, wherein NRR¹ is

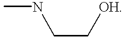

An eleventh embodiment of a first aspect of the present invention is a compound of the sixth embodiment, including pharmaceutically acceptable salts thereof, wherein W is hydrogen; R² is hydroxy; and R³ is hydrogen.

A twelth embodiment of a first aspect of the present invention is a compound of the eleventh embodiment, including pharmaceutically acceptable salts thereof, wherein NRR¹ is selected from the group consisting of:

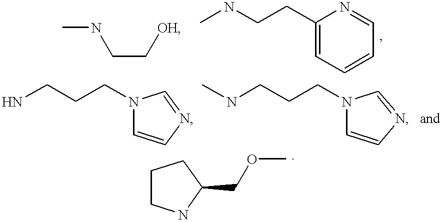

A thirteenth embodiment of a first aspect of the present invention is a compound of the third embodiment, including pharmaceutically acceptable salts thereof, wherein Y is S(O)ₘ in which m is 0 or 2;

R¹ is selected from the group consisting of CH₃, CH₂CH₃, (CH₂)₂OH, CH(CH₃)CH₂OH, CH₂[CH(OH)]₄CH₂OH, [(CH₂)₂O]₂(CH₂)₂OH, [(CH₂)₂O]₂(CH₂)₂OCH₃, [(CH₂)₂O]₂(CH₂)₂NH₂, [(CH₂)₂O]₂(CH₂)₂N(CH₃)₂, CH₂CO₂H, (CH₂)₂CO₂H, CH(CO₂H)CH₂CO₂H, CH₂CH(NHC(O)CH₃)CO₂H, (CH₂)₂SO₃H, (CH₂)₄NH₂, (CH₂)₂N(CH₃)₂, (CH₂)₃N(CH₃)₂, (CH₂)₂N(CH₂CH₃)₂, (CH₂)₂NH(CH₃), (CH₂)₂NH(CH₂CH₃), (CH₂)₂NH(CH₂)₂OH, (CH₂)₂N[(CH₂)₂NH₂)]₂, (CH₂)₂NHC(O)CH₃,

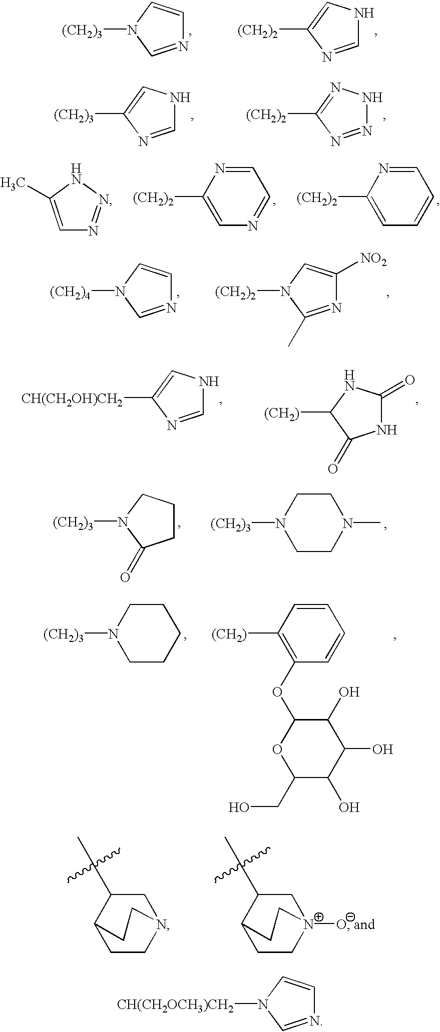

A fourteenth embodiment of a first aspect of the present invention is a compound of the thirteenth embodiment, including pharmaceutically acceptable salts thereof, wherein m is 0; and W is hydrogen or

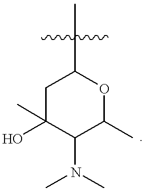

A fifteenth embodiment of a first aspect of the present invention is a compound of the fourteenth embodiment, including pharmaceutically acceptable salts thereof, wherein W is

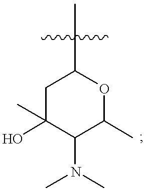

R² is hydroxy; and R³ is hydrogen.

A sixteenth embodiment of a first aspect of the present invention is a compound of the fifteenth embodiment, including pharmaceutically acceptable salts thereof, wherein R¹ is selected from the group consisting of CH₂CO₂H, (CH₂)₂CO₂H, CH(CO₂H)CH₂CO₂H, CH₂CH(NHC(O)CH₃)CO₂H, (CH₂)₂SO₃H, (CH₂)₂N(CH₃)₂, (CH₂)₂N(CH₂CH₃)₂,

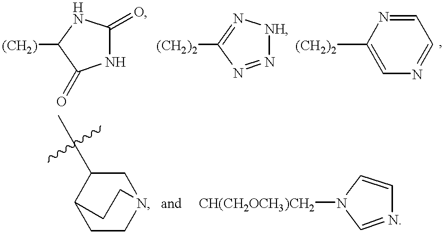

A seventeenth embodiment of a first aspect of the present invention is a compound of the fourteenth embodiment, including pharmaceutically acceptable salts thereof, wherein W is hydrogen; R² is hydroxy; and R³ is hydrogen.

An eighteenth embodiment of a first aspect of the present invention is a compound of the seventeenth embodiment, including pharmaceutically acceptable salts thereof, wherein R¹ is CH₂CO₂H or (CH₂)₂N(CH₂CH₃)₂.

A nineteenth embodiment of a first aspect of the present invention is a compound of the thirteenth embodiment, including pharmaceutically acceptable salts thereof, wherein m is 2; and W is

[chemical structure]

A twentieth embodiment of a first aspect of the present invention is a compound of the nineteenth embodiment, including pharmaceutically acceptable salts thereof, wherein R² is hydroxy; and R³ is hydrogen.

A twentyfirst embodiment of a first aspect of the present invention is a compound of the twentieth embodiment, including pharmaceutically acceptable salts thereof, wherein R¹ is selected from the group consisting of:

(CH₂)₂N(CH₂CH₃)₂,

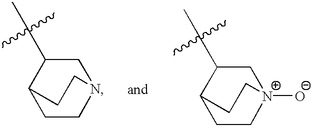

A twentysecond embodiment of a first aspect of the present invention is a compound of the first embodiment, including pharmaceutically acceptable salts thereof, wherein Q is

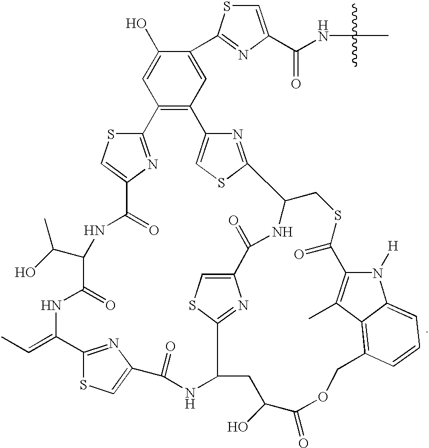

A twentythird embodiment of a first aspect of the present invention is a compound of the twentysecond embodiment, including pharmaceutically acceptable salts thereof, wherein Y is NR.

A twentyfourth embodiment of a first aspect of the present invention is the compound of the twentythird embodiment, including pharmaceutically acceptable salts thereof, wherein R is methyl; and R¹ is 3-(imidazol-1-yl)-propyl.

A twentyfifth embodiment of a first aspect of the present invention is a compound of the twentysecond embodiment, including pharmaceutically acceptable salts thereof, wherein Y is S.

A twentysixth embodiment of a first aspect of the present invention is the compound of the twentyfifth embodiment, including pharmaceutically acceptable salts thereof, wherein R¹ is (CH₂)₂N(CH₂CH₃)₂.

A first embodiment of a second aspect of the present invention is a pharmaceutical composition which comprises a therapeutically effective amount of a compound as described in any of the embodiments of the first aspect, and a pharmaceutically acceptable carrier, adjuvant or diluent.

A first embodiment of a third aspect of the present invention is a method of treating or preventing bacterial or mycobacterial infection by administering to a mammal in need thereof a therapeutically effective amount of a compound as described in any of the embodiments of the first aspect.

A second embodiment of the third aspect of the present invention is the method of the first embodiment, wherein said bacterial infection is caused by a gram positive bacteria or a mycobacterium.

A third embodiment of a third aspect of the present invention is the method of the second embodiment, wherein said gram positive bacterial infection or mycobacterial infection is caused by methicillin-resistant Staphylococcus aureus, vancomycin-resistant Staphylococcus aureus, vancomycin-resistant Enterococcus faecalis, vancomycin-resistant Enterococcus faecium or Mycobacteria tuberculosis.

An “aryl” group refers to an all carbon monocyclic or fused-ring polycyclic (i.e., rings which share adjacent pairs of carbon atoms) groups having a completely conjugated pi-electron system. Examples, without limitation, of aryl groups are phenyl, napthalenyl and anthracenyl. The aryl group may be substituted or unsubstituted as specified.

As used herein, a “heteroaryl” group refers to a monocyclic or fused ring (i.e., rings which share an adjacent pair of atoms) group having in the ring(s) one or more atoms selected from the group consisting of nitrogen, oxygen and sulfur and, in addition, having a completely conjugated pi-electron system. Examples, without limitation, of heteroaryl groups are furyl, thienyl, benzothienyl, thiazolyl, imidazolyl, oxazolyl, oxadiazolyl, thiadiazolyl, benzthiazolyl, triazolyl, tetrazolyl, isoxazolyl, isothiazolyl, pyrrolyl, pyranyl, tetrahydropyranyl, pyrazolyl, pyridinyl, pyrimidinyl, quinolinyl, isoquinolinyl, purinyl, carbazolyl, benzoxazolyl, benzimidazolyl, indolyl, isoindolyl, and pyrazinyl. The heteroaryl group may be substituted or unsubstituted as specified.

As used herein, a “heteroalicyclic” group refers to a monocyclic or fused ring group having in the ring(s) one or more atoms selected from the group consisting of nitrogen, oxygen and sulfur. The rings may also have one or more double bonds. However, the rings do not have a completely conjugated pi-electron system. Examples, without limitation, of heteroalicyclic groups are azetidinyl, piperidinyl, piperazinyl, imidazolinyl, thiazolidinyl, pyrrolidinyl, aziridinyl, morpholinyl, thiomorpholinyl and tetrahydropyranyl. The heteroalicyclic group may be substituted or unsubstituted as specified.

An “alkyl” group refers to a saturated aliphatic hydrocarbon including straight chain and branched chain groups. Preferably, the alkyl group has 1 to 20 carbon atoms (whenever a numerical range; e.g., “1-20”, is stated herein, it means that the group, in this case the alkyl group may contain 1 carbon atom, 2 carbon atoms, 3 carbon atoms, etc. up to and including 20 carbon atoms). For example, the term “C₁₋₆alkyl” as used herein and in the claims (unless specified otherwise) mean straight or branched chain alkyl groups such as methyl, ethyl, propyl, isopropyl, butyl, isobutyl, t-butyl, amyl, hexyl and the like. More preferably, it is a medium size alkyl having 1 to 10 carbon atoms. The alkyl group may be substituted or unsubstituted as specified. When substituted, the substituent group(s) may include, for example, one or more individually selected from aryl, heteroaryl, heteroalicyclic, hydroxy, alkoxy, amino, etc.

A “cycloalkyl” group refers to an all-carbon monocyclic or bicyclic ring system. Examples, without limitation, of cycloalkyl groups are cyclopropane, cyclobutane, cyclopentane, cyclohexane, cycloheptane and adamantane.

A “hydroxy” group refers to an —OH group.

An “alkoxy” group refers to an —O-alkyl group as defined herein.

An “amino” group refers to an —NH₂ group. One or both of the hydrogens attached to the amino nitrogen can be replaced by alkyl goups to provide an alkylamino or di(alkyl)amino group, respectively.

It is known in the art that nitrogen atoms in heteroaryl systems can be “participating in a heteroaryl ring double bond”, and this refers to the form of double bonds in the two tautomeric structures which comprise five-member ring heteroaryl groups. This dictates whether nitrogens can be substituted as well understood by chemists in the art. The disclosure and claims of the present invention are based on the known general principles of chemical bonding.

Physiologically acceptable salts of compounds disclosed herein are within the scope of this invention. The term “pharmaceutically acceptable salt” as used herein and in the claims is intended to include nontoxic base addition salts. Suitable salts include those derived from organic and inorganic acids such as, and without limitation, hydrochloric acid, hydrobromic acid, phosphoric acid, sulfuric acid, methanesulfonic acid, acetic acid, tartaric acid, lactic acid, sulfinic acid, citric acid, maleic acid, fumaric acid, sorbic acid, aconitic acid, salicylic acid, phthalic acid, and the like. The term “pharmaceutically acceptable salt” as used herein is also intended to include salts of acidic groups, such as a carboxylate, with such counterions as ammonium, alkali metal salts, particularly sodium or potassium, alkaline earth metal salts, particularly calcium or magnesium, and salts with suitable organic bases such as lower alkylamines (methylamine, ethylamine, cyclohexylamine, and the like) or with substituted lower alkylamines (e.g. hydroxyl-substituted alkylamines such as diethanolamine, triethanolamine or tris(hydroxymethyl)-aminomethane), or with bases such as piperidine or morpholine.

The compounds of Formula I may exist as single diastereomers or as mixtures thereof. The general formula I is intended to encompass single diastereomers such as those depicted below as I′ and I″ and any mixtures thereof. Single diastereomers may be obtained from diastereomeric mixtures by methods such as preparative HPLC as described hereinafter.

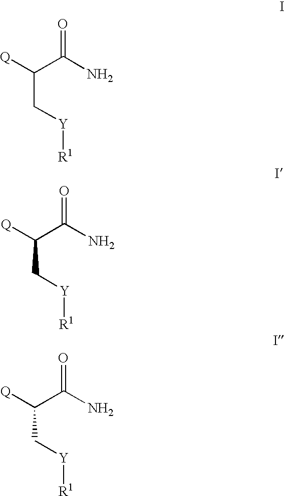

The compounds of the present invention may have chiral centers other than those centers whose stereochemistry is depicted in Formula I, and therefore may occur as mixtures of diastereomers or as single diastereomers. It is understood that all such isomeric forms, and any mixtures thereof, are included in the present invention. For example, when the group W in a compound of Formula I is a sugar residue of the formula

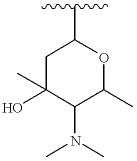

it is to be understood to encompass racemic forms of the sugar residue as well as chiral forms of the sugar residue such as

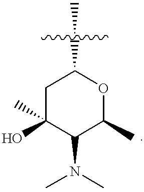

As an additional example, in a compound of Formula I when Y is NR, the moiety NRR¹ could be a group such as 2-hydroxymethyl pyrrolidinyl,

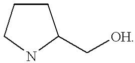

It is to be understood that the 2-hydroxymethyl pyrrolidinyl group shown encompasses both racemic as well as chiral forms of the 2-hydroxymethyl pyrrolidinyl group. In addition, some of the compounds of the present invention may form solvates with water or common organic solvents. Such solvates are within the scope of this invention.

The following abbreviations, most of which are conventional abbreviations well known to those skilled in the art, are used throughout the description of the invention and examples. Some of the abbreviations used are as follows:

h=hour(s)

rt=room temperature

mol=mole(s)

mmol=millimole(s)

g=gram(s)

mg=milligram(s)

CH₂Cl₂=Dichloromethane

DMF=N,N-Dimethylformamide

DMSO=Dimethylsulfoxide

Et₃N=Triethylamine

HCl=Hydrochloric Acid

MCPBA=3-Chloroperoxybenzoic acid

NMP=1-Methyl-2-pyrrolidinone

THF=Tetrahydrofuran

TFA=Trifluoroacetic acid

The general procedures used to synthesize the novel thiazolyl peptide compounds within Formula I are depicted in Reaction Schemes 1-4, below. Reaction Scheme 1 shows the preparation of compounds of Formula I by the Michael type addition of an appropriate nucleophile, HYR¹, to an appropriately substituted and activated carbon-carbon double bond of a thiazolyl peptide antibiotic such as a nocathiacin or nosiheptide.

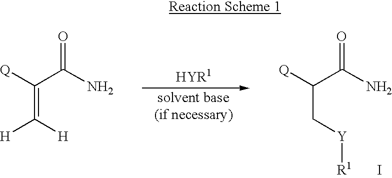

The reaction is typically run in a polar solvent and may require the presence of an organic base. Appropriate nucleophiles of formula HYR¹ include primary amines, secondary amines and thiols. Thiazolyl peptide antibiotics which may be employed as starting materials for the preparation of compounds of Formula I, include any thiazolyl peptide antibiotic which contains a carbon-carbon double bond which can act as a Michael acceptor for the Michael donor, HYR¹. Thiazolyl peptide antibiotics, including, but not limited to, nocathiacins, nosiheptide, thiostrepton, thiopeptin Ba, siomycins, micrococcin, SCH 40832, S-54832, and GE-37468 may be appropriate starting materials for the Michael type additions described herein. The thiazolyl peptide nocathiacins and nosiheptide serve as precursors to compounds of Formula I. It is also understood that certain thiazolyl peptide antibiotics contain more than one carbon—carbon double bond which can act as a Michael acceptor. In such cases, it is understood that between one and all of the thiazolyl peptide Michael acceptor carbon—carbon double bonds can react with the nucleophile, HYR¹, to provide compounds in a similar fashion to those of Formula I. The synthesis of the compounds of Formula I is typically carried out in a polar solvent including, but not limited to, water, DMF, DMSO, dioxane, THF, CH₂Cl₂, NMP, methanol, ethanol, propanol, butanol and any mixtures thereof. A preferred solvent for reactions which employ nocathiacins as the starting material is water. Reactions which employ nosiheptide or thiostrepton as starting materials are preferably carried out in a mixture of DMF and water. Certain reactions require the addition of an organic base, such as triethylamine, N,N-diisopropylethylamine, 4-methylmorpholine, 1,8-Diazabicyclo[5.4.0]undec-7-ene (DBU), 1,5-Diazabicyclo[4.3.0]non-5-ene (DBN), 1,4-Diazabicyclo[2.2.2]octane (Dabco) or pyridine. Triethylamine is a preferred base, when a base is required. The Michael addition reactions, as shown in Reaction Scheme 1, may be carried out within a temperature range of −50° C. to 50° C. A preferred method is to initiate the reaction at room temperature and then maintain the reaction mixture within the temperature range of −20° C. to 0° C. for a period of 3 to 24 hours. It is understood by one skilled in the art that certain reactions, such as reactions employing sterically hindered nucleophiles, may require extended reaction times outside the typical period of 3 to 24 hours.

[chemical structure]

Reaction Scheme 1A depicts the preparation of a nocathiacin derivative within Formula I. The nocathiacin starting material is treated with an appropriate amine or thiol of general formula HYR¹ in water and in the presence of a tertiary amine base, such as triethylamine, if necessary.

Reaction Scheme 2 depicts the preparation of compounds of Formula Ia, Ia′ and Ia″ wherein the Michael donor (i.e. nucleophile) employed is a thiol. The presence of an organic base, preferably triethylamine, is required when a thiol of general formula R¹—SH is used as the Michael donor. The sulfide compounds of Formula Ia which are formed as shown in the first step of Scheme 2 can then be oxidized to provide sulfoxide (m=1) of Formula Ia′ and sulfone (m=2) of Formula Ia″

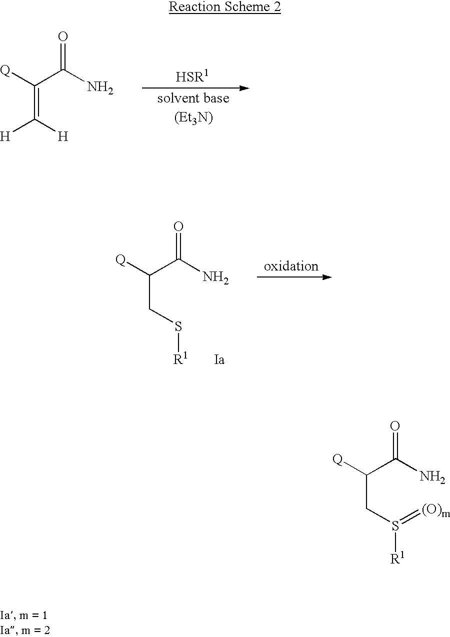

derivatives as shown in the second step of Scheme 2. Oxidation of the sulfide to the corresponding sulfoxide (m=1) may be accomplished by treatment with mCPBA in the presence of either TFA or sodium bicarbonate as described by J. C. Barriere and J. M. Paris, ‘From the Michael Reaction to the Clinic.’ In: Anti-infectives: Recent Advances in Chemistry and Structure-Activity Relationships. Bentley, P. H., O'Hanlon, P. J. eds, The Royal Society of Chemistry, Cambridge, 1997, Chpt. 3, p 36. Oxidation of the sulfide to the sulfone (m=2), shown in Scheme 2, may be accomplished by methods as described at p 37-38 of the preceding reference and further described in Chaterjee, D.; Harris, N. V.; Parker, T; Smith, C.; Warren, J. P. Eur. Patent Appl. 252,720 (use of sodium periodate and ruthenium trichloride) and Radisson, X; World Patent WO 92/01692-A, 1992 (use of hydrogen peroxide with sodium tungstate).

Reaction Scheme 3 depicts the preparation of compounds of Formula Ib wherein the Michael donor employed is a primary or secondary amine of the general formula HNRR¹. The presence of an organic base, preferably triethylamine, may be required when certain amines are used.

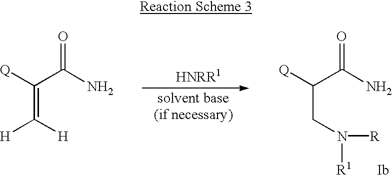

Reaction Scheme 4, below, depicts further derivatization of compounds of Formula I which possess a reactive amine residue. The amine residue can react with isocyanates, such as methyl isocyanate, of general formula R′NCO (wherein R′ typically represents a C₁₋₆alkyl) to provide urea derivatives as shown in equation 1 of Scheme 4. Alternatively, reaction with anhydrides of general formula (R′CO)₂O (wherein R′ typically represents a C₁₋₆alkyl or both R′ groups taken together represent a C₂₋₄alkylene) provides amide derivatives as shown in equation 2 of Scheme 4. Useful anhydrides include, but are not limited to, acetic anhydride and succinic anhydride.

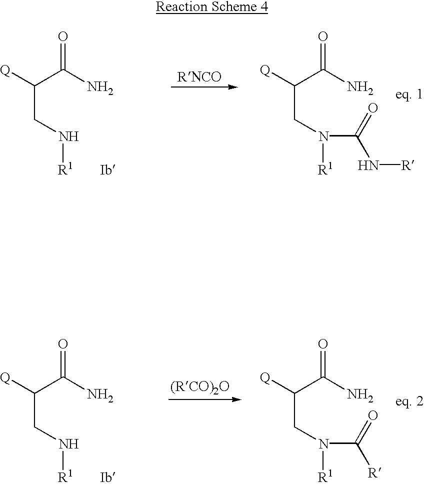

### Starting Materials

The nocathiacin antibiotics and nosiheptide may be employed as starting materials for the synthesis of compounds of Formula I. The other thiazolyl peptide antibiotics disclosed may undergo the Michael type addition of a nucleophile of the general formula HYR¹ in an analogous fashion.

The nocathiacin starting materials were prepared by fermentation using microorganism ATCC-202099 and subsequent isolation as described by J. E. Leet et al. in PCT Appl. WO 00/03722 (published Jan. 27, 2000); and PCT Appl. WO 00/14100 and also by Sasaki, T. et al. in J. of Antibiotics 1998, 51, No. 8, pp. 715-721. The preparation of nocathiacin I and III (see structure below) is described more fully in commonly-owned U.S. Pat. No. 6,218,398 (issued Apr. 17, 2001) which is hereby incorporated by reference in its entirety. The groups W, X, R³ and R⁴ are as defined in the specification. For nocathiacin I (see general structure below), R² is OH, R³ is H, and W is

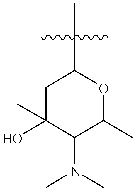

whereas for nocathiacin III, R² is OH; W and R³ are H. For the nocathiacin derivative designated MJ347-81F4-B, the preparation of which is described by Sasaki, T. et al. in J. of Antibiotics 1998, 51, No. 8, pp. 715-721, R² is OH, R⁴ is H, and W is

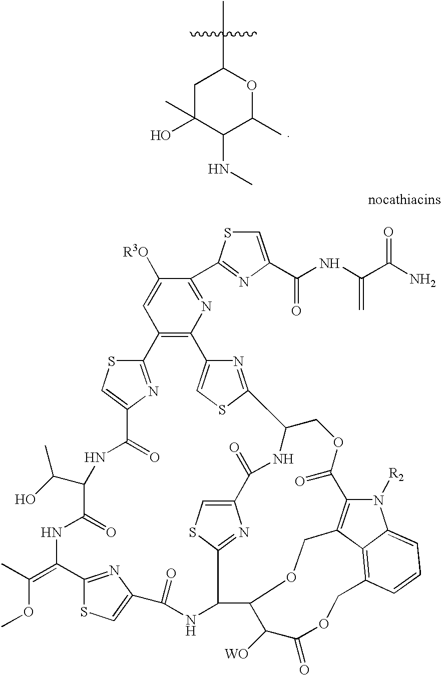

Nosiheptide can be prepared and isolated according to methods described in Pascard, C. et al. in J. Am. Chem. Soc. 1977, 99, 6418-6423 and Benazet, F. et al. in Experientia 1980, 36, 414-416.

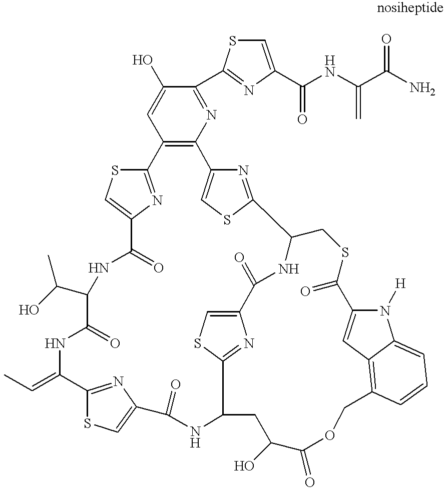

Thiostrepton is a fermentation product which is produced and isolated from Streptomyces azureus as described by Vandeputte, J.; and Dutcher, J. D. in Antibiotics Ann. 1956, 560 and also in Pagano, J. F. et al. Antibiotics Ann. 1955-1956, 554-559.

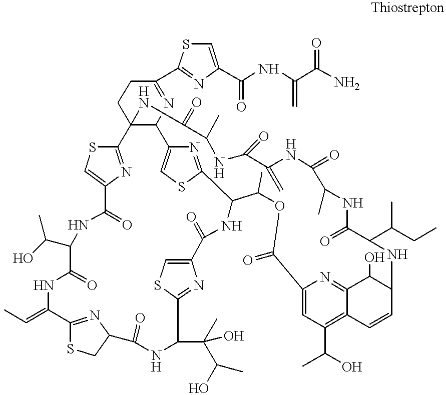

Antibiotic S 54832 is produced by fermentation using a Micromonospora globosa strain and isolated according to the methods described by Keller-Juslen, C.; Kuhn, M.; and King, H. D. in U.S. Pat. No. 4,478,831 (1984).

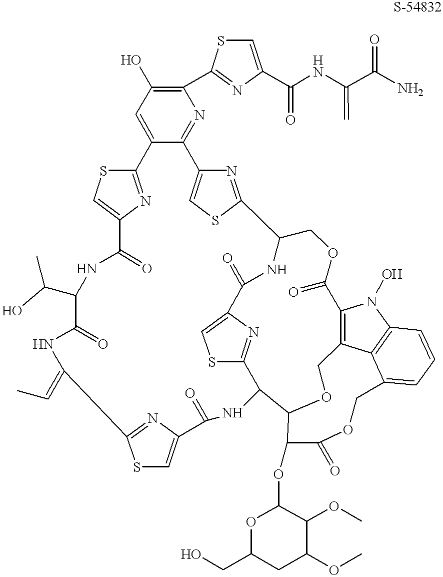

Antibiotic GE37468 (shown below) may be produced by fermentation using Streptomyces sp. Strain ATCC 55365 and isolated according to procedures disclosed by Stella, S. et al in J. of Antibiotics 1995, 48, No. 8, 780-786.

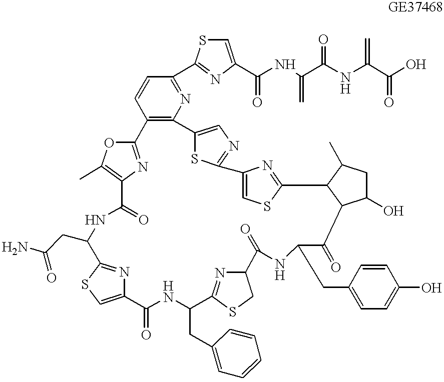

Methylsulfomycin I is produced by fermentation using Streptomyces sp. HIL Y-9420704 according to procedures disclosed by Kumar et al. in J. Nat. Prod. 1999, 62(11), 1562-1564.

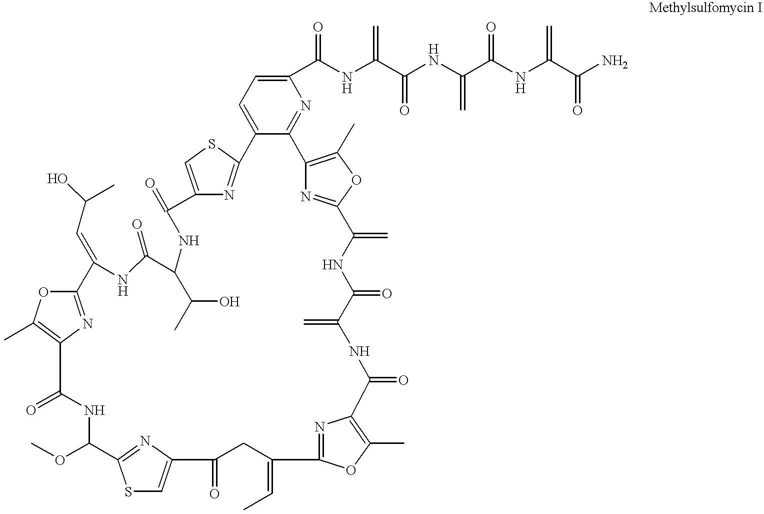

Sulfomycin can be prepared by fermentation and isolated as described within J. Am. Chem. Soc. 1996, 118, 11363.

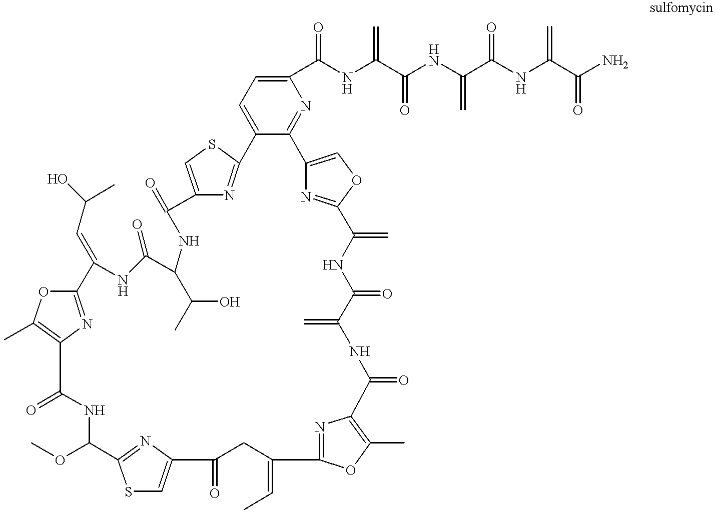

Micrococcin can also be obtained by fermentation and isolated as described in Antibiotics 1975, 3, 480.

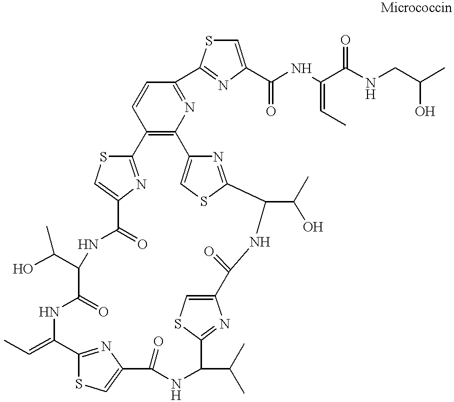

Promothiocin A (wherein R=H) and Promothiocin B (wherein R=C(═CH₂)CONHC(═CH₂)CONH₂) can be prepared by fermentation according to

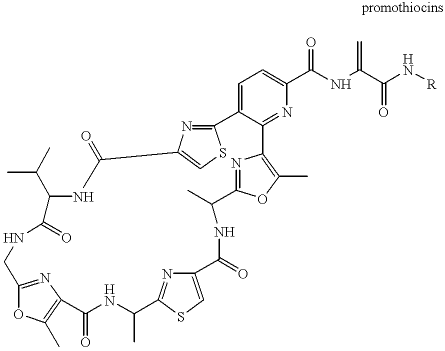

the methods described by Yun, B. S. et al. in J. Antibiotics 1994, 47, 510-514 or by total synthesis according to the methods described by Bagley, B. C. et al. in J. Am. Chem. Soc. 2000, 122, 3301-3313.

### Synthesis

The synthesis of the compounds of Formula I was carried out by standard methods practiced by one skilled in the art. Purification of the compounds of Formula I by medium pressure liquid chromatography (MPLC) was performed using Waters Preparative C-18 reverse phase material, 125 Å, 55-105 μm which was packed in a Michel-Miller column. The mobile phase was pumped through the packed Michel-Miller column using an FMI Lab Pump (Model RP-SY, Fluid Metering Inc., Oyster Bay, N.Y.) at system pressures generally within the range of 20-80 psi. Typical eluents used for MPLC include mixtures of acetonitrile and water or methanol and water with the pH of these eluents sometimes being adjusted to 4-5 by the addition of dilute aqueous HCl or TFA. The compounds of the present invention could also be purified by standard reverse phase column chromatography using Waters Preparative C-18 reverse phase material, 125 Å, 55-105 μm and acetonitrile and water or methanol and water as eluents, with the pH of these eluents sometimes being adjusted to 4-5 by the addition of dilute aqueous HCl or TFA. Compounds purified by preparative HPLC were typically diluted in water, DMF, MeOH or some mixture thereof and purified on a Shimadzu LC-10A automated preparative HPLC system. A representative HPLC method useful for the purification of compounds of Formula I is given below.

General Preparative HPLC Method (i.e., compound purification)

Purification Method: Initial gradient (20% B, 80% A) ramp to final gradient (100% B, 0% A) over 20 minutes, hold for 3 minutes (100% B, 0%A)

Solvent A: 10% MeOH/90% H₂O/0.1% Trifluoroacetic Acid

Solvent B: 10% H₂O/90% MeOH/0.1% Trifluoroacetic Acid

Column: YMC C18 S5 20×100 mm column

Detector Wavelength: 220 nm

The compounds of Formula I purified by preparative HPLC were obtained as trifluoroacetic acid salts which could be converted to the corresponding hydrochloride salt using AG 1-X2 Resin, 100-200 mesh, chloride form, obtained from BioRad (Hercules, Calif.). The AG 1-X2 resin was placed in a fritted syringe and washed with water. An aqueous solution of a trifluoroacetic acid salt of a compound of Formula I was then filtered through the resin. The resin was then rinsed with three volumes of water. The combined aqueous fractions were frozen and lyophilized to provide the compound of Formula I as a hydrochloride salt. NMR spectra were recorded on a Bruker DPX-500, DPX-300 or Varian XL-300. Chemical shifts (δ) are expressed in ppm. Coupling constants (J) are expressed in Hertz. Mass spectra (MS) for the compounds were obtained using Flow Injection Mass Spectrometry (electrospray ionization technique) on a Finnegan SSQ 7000 mass spectrometer. Liquid Chromatography/ Mass Spectrometry (LC/MS) was performed on a Hewlett-Packard 1100 liquid chromatograph coupled to a Finnegan LCQ mass spectrometer using an electrospray ionization technique. High Resolution Mass Spectrometry (HRMS) for the compounds was obtained using a Finnegan MAT 900 mass spectrometer with 5000 resolution at 10% valley.

General Procedure for the Michael Addition of Amines to a Thiazolyl Peptide Antibiotic

To a stirred solution of an appropriate amine (10 equiv.) in an appropriate solvent, (C=0.002 M) at room temperature was added an appropriate thiazolyl peptide antibiotic (1 equiv.). The reaction mixture was stirred for 3 to 5 minutes until the reaction mixture becomes a clear homogeneous solution. If the reaction mixture did not become a clear solution then enough Et₃N, (˜2-4 equiv.) was added dropwise and stirred until the reaction mixture becomes a clear solution. The resulting reaction mixture was maintained at approximately −20° C. for a period of 3 to 24 h. After the reaction was complete, the reaction mixture was concentrated under reduced pressure or it was quenched by addition of TFA or HCl (1N) until the pH of the mixture was acidic and then concentrated under reduced pressure. The residue was then dissolved in a suitable solvent such as water, DMF, methanol or a mixture thereof and was purified using Prep-HPLC and/or MPLC on preparative C-18 column using methanol/water or acetonitrile water as eluent. The eluent may contain dilute HCl or TFA (such that the pH is 4-5). Alternatively, the reaction mixture can be directly subjected to purification by HPLC or MPLC or quenched with HCl or TFA and purified by HPLC or MPLC. The fractions containing the desired product as a trifluoroacetic acid, or hydrochloride salt were combined and concentrated to approximately 50 mL and then freeze dried to give products as yellow fluffy solids.

General Procedure for the Michael Addition of Thiols to a Thiazolyl Peptide Antibiotic

To a stirred suspension of an appropriate thiazolyl peptide antibiotic (0.20 mmol) in an appropriate solvent (10 mL) was added Et₃N (70 μL, 0.5 mmol) and continued stirring at room temperature for 3 to 5 minutes until the reaction mixture becomes a clear homogeneous solution. To this solution was added an appropriate thiol (5-10 equiv.) and, if needed, more Et₃N (usually 2-3 mmol) to bring the reaction mixture to a homogeneous solution. The reaction mixture was then maintained at −20° C. for a period of 3 to 24 h. The reaction mixture was then diluted with water, aqueous sodium bicarbonate or dilute hydrochloric acid and purified using Prep-HPLC or MPLC on preparative C-18 column using methanol/water or acetonitrile/water as eluent. The eluent may contain dilute HCl or TFA (such that the pH is 4-5). The fractions containing the desired product as a trifluoroacetic acid, triethylammonium, sodium or hydrochloride salt were combined and concentrated to approximately 50 mL and then freeze dried to give products as yellow fluffy solids.

When the compounds of Formula I are employed as pharmaceutical compositions for the treatment of bacterial infections, they may be combined with one or more pharmaceutically acceptable carriers, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solutions or suspension containing from about 0.05 to 5% suspending agent in an isotonic medium. Such pharmaceutical preparations may contain, for example, from about 0.05 up to about 90% of the active ingredient in combination with the carrier, more usually between about 5% and 60% by weight.

The effective dosage of active ingredient employed may vary depending on the particular compound employed, the mode of administration and the severity of the condition being treated. However, in general, satisfactory results are obtained when the compounds of the invention are administered at a daily dosage of from about 0.5 to about 500 mg/kg of animal body weight, preferably given in divided doses two to four times a day, or in sustained release form. For most large mammals the total daily dosage is from about 1 to 100 mg, preferably from about 2 to 80 mg. dosage forms suitable for internal use comprise from about 0.5 to 500 mg of the active compound in intimate admixture with a solid or liquid pharmaceutically acceptable carrier. This dosage regimen may be adjusted to provide the optimal therapeutic response. For example, several divided doses may be administered daily or the dose may be proportionally reduced as indicated by the exigencies of the therapeutic situation.

These active compounds may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

These active compounds may also be administered parenterally or intraperitoneally. Solutions or suspensions of these active compounds as a free base or pharmacologically acceptable salt can be prepared in water suitably mixed with a surfactant such as hydroxypropylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

### EXAMPLES

The following examples set out the preparation of the novel thiazolyl peptide derivatives and their biological properties. Reasonable variations, such as those which would occur to a skilled artisan, can be made herein without departing from the scope of the invention.

### Compounds of Formula I

### Example 1

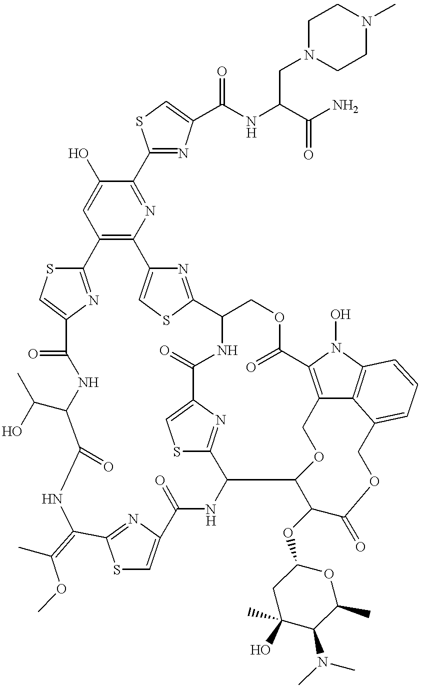

An example of the general procedure for the Michael addition of amines to a Nocathiacin: To a stirred solution of 1-methylpiperazine (0.38 mL, 3.50 mmol) (10 equiv.) in water (17.5 mL) at room temperature was added Nocathiacin I (0.5 g, 0.35 mmol) (1 equiv.). The reaction mixture was stirred for 3 to 5 minutes until it became a clear homogeneous solution. The reaction mixture was then maintained at −20° C. for 16 h. The reaction mixture was then quenched with 1 N aqueous hydrochloric acid and subjected to medium pressure chromatography, as described above. The product containing fractions were concentrated under reduced pressure, frozen and lyophilized to afford the product as a yellow powder (0.223 g, 41% yield). ¹H NMR (500 MHz, DMSO-d₆): δ 11.03 (bs, 1H), 10.79 (s, 1H), 10.18 (bs, 1H), 9.12 (s, 1H), 9.02 (m, 1H), 8.71 (m, 1H), 8.65 (s, 1H), 8.59 (d, J=10.0 Hz, 1H), 8.54 (s, 1H), 8.51 (s, 1H), 8.23 (s, 1H), 8.03 (s, 1H), 7.89 (d, J=5.0 Hz, 1H), 7.86 (d, J=10.0 Hz, 1H), 7.74 (d, J=10.0 Hz, 1H), 7.63 (s, 1H), 7.36 (m, 2H), 7.23 (s, 1H), 7.09 (d, J=10.0 Hz, 1H), 6.02 (d, J=10.0 Hz, 1H), 5.75 (m, 1H), 5.71 (d, J=10.0 Hz, 1H), 5.22 (d, J=5.0 Hz, 1H), 5.05 (m, 3H), 4.79 (d, J=10.0 Hz, 1H), 4.66 (m, 1H), 4.53 (d, J=10.0 Hz, 1H), 4.30 (d, J=10.0 Hz, 1H), 4.25 (m, 1H), 4.15 (d, J=10.0 Hz, 1H), 4.05 (d, J=10.0 Hz, 1H), 3.19 (m, 4H), 3.45-3.25 (m, 6H), 3.12-2.80 (m, 7H), 2.73 (m, 2H), 2.54 (s, 2H), 2.12 (m, 1H), 2.00 (s, 3H), 1.93 (d, J=15.0 Hz, 1H), 1.60 (s, 3H), 1.16 (m, 3H), 0.80 (d, J=10.1 Hz, 3H). MS: 1538.1 (M+H)⁺, 1535.5 (M-H)⁻; HRMS (ES) calcd. for C₆₆H₇₃N₁₆O₁₈S₅ (M+H)⁺: 1537.389, found: 1537.386. Anal. Calcd. for C₆₆H₇₂N₁₆O₁₈S₅2HCl6H₂O: C, 46.12; H, 5.04; N, 13.04; S, 9.33; Cl, 4.13. Found: C, 46.33; H, 4.98; N, 12.82; S, 9.55; Cl, 4.04.

### Example 2

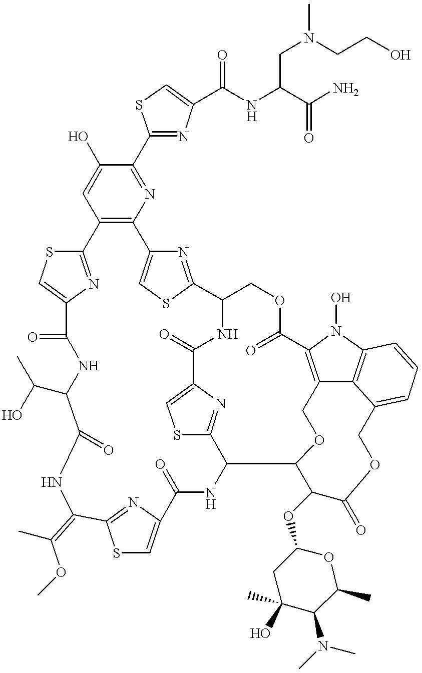

Following the procedure as described in Example 1 using Nocathiacin I (0.5 g, 0.35 mmol) and 2-(methylamino)ethanol (0.28 mL, 3.50 mmol) in water (17.5 mL). The reaction mixture was then quenched with 1N aqueous hydrochloric acid and subjected to medium pressure chromatography. The product containing fractions were concentrated under reduced pressure, frozen and lyophilized to afford the product as a yellow powder (0.30 g, 57% yield). ¹H NMR (500 MHz, DMSO-d₆): δ 10.95 (dd, J=8.33, J=17.50, 1H), 10.78 (s, 1H), 9.60-9.35 (m, 2H), 9.12 (s, 1H), 8.75 (bs, 1H), 8.66 (s,1H), 8.59 (s, 2H), 8.54 (s,1H), 8.23 (s,1H), 8.05 (s, 1H), 7.93 (d, J=18.2 Hz, 1H), 7.85 (m, 2H), 7.81 (d, J=9.9 Hz, 1H), 7.54 (d, J=18.0 Hz, 1H), 7.35 (m, 2H), 7.19 (d, J=9.8 Hz, 1H), 6.40 (s, 1H), 6.03 (d, J=15.0 Hz, 1H), 5.75 (m, 1H), 5.71 (d, J=14.9 Hz, 1H), 5.33 (bs, 1H), 5.21 (d, J=20.0 Hz, 1H), 5.05 (m, 3H), 5.00 (m, 1H), 4.72 (d, J=14.7 Hz, 1H), 4.52 (d, J=14.8 Hz, 1H), 4.30 (d, J=10.1 Hz, 1H), 4.25 (m, 1H), 4.15 (d, J=9.8 Hz, 1H), 4.05 (d, J=10.0 Hz, 1H), 3.94-3.85 (m, 4H), 3.77 (m, 2H), 3.72-3.61 (m, 2H), 3.39 (m, 4H), 3.17, (s, 4H), 3.12 (s, 1H), 2.89 (m, 1H), 2.73 (s, 2H), 2.54 (m, 1H), 2.13, (m, 1H), 2.00 (s, 3H), 1.93 (d, J=9.9 Hz, 1H), 1.61 (s, 3H), 1.15 (s, 3H), 0.80 (d, J=6.9 Hz, 3H). MS: 1512.4 (M+H)⁺, 1511.6 (M−H)⁻; HRMS (ES) calcd. for C₆₄H₇₀N₁₅O₁₉S₅ (M+1): 1512.358, found: 1512.358. Anal. Calcd. for C₆₄H₆₉N₁₅O₁₉S₅2HCl4H₂O: C, 46.37; H, 4.80; N, 12.67; S, 9.67; Cl, 4.28. Found: C, 46.45; H, 4.76; N, 12.77; S, 9.84; Cl, 4.69.

### Example 3

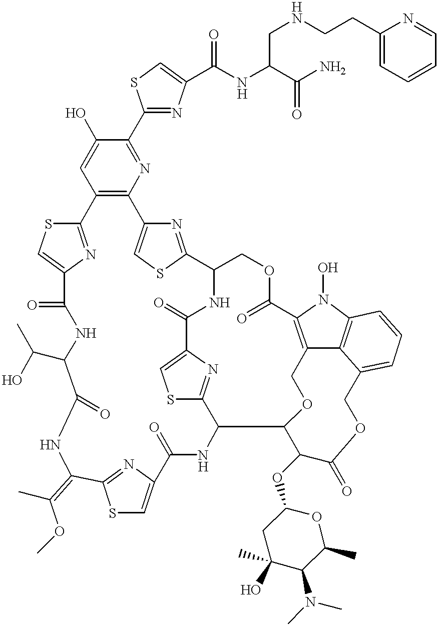

Following the procedure described in Example 1 using Nocathiacin I (0.5 g, 0.35 mmol) and 2-(2-aminoethyl)pyridine (0.42 mL, 3.50 mmol) in water (17.5 mL) afforded the product as a yellow powder (0.25 g, 46% yield). ¹H NMR (500 MH z, MeOD): δ 10.73 (bs, 1H), 9.29 (bs, 1H), 9.12 (s, 1H), 8.66 (s, 1H), 8.60 (d, J=8.0 Hz, 1H), 8.55 (s, 1H), 8.52 (s, 1H), 8.42 (t, 1H), 8.27 (s, 1H), 8.02 (s, 1H), 7.91 (s, 1H), 7.88 (d, J=11.1 Hz, 1H), 7.73 (m, 3H), 7.48 (m, 1H), 7.34 (m, 3H), 7.23 (m, 1H), 7.18 (d, J=7.0 Hz, 1H), 6.00 (d, J=12.1 Hz, 1H), 5.74 (m, 1H), 5.68 (d, J=9.6 Hz, 1H), 5.22 (m, 1H), 5.07 (m, 2H), 4.94 (d, J=4.4 Hz, 1H), 4.85 (m, 1H), 4.76 (d, J=10.3 Hz, 1H), 4.50 (d, 11.1, 1H), 4.31 (d, J=9.7 Hz, 1H), 4.26 (t, J=5.20 Hz, 1H), 4.10 (d, J=10.5 Hz, 1H), 4.04 (d, J=11.1 Hz, 1H), 3.91 (s, 3H), 3.76 (m, 1H), 3.60-3.23 (m, 12H), 3.09 (bs, 2H), 2.60-2.38 (m, 5H), 2.09 (m, 1H), 1.99 (s, 2H), 1.96 (m, 1H), 1.80 (d, J=14.0 Hz, 1H), 1.43 (s, 3H), 1.25-1.09 (m, 3H), 0.57 (d, J=6.0 Hz, 3H). MS: 1559.7 (M+H)⁺, 1558.5 (M); HRMS (ES) calcd. for C₆₈H₇₀N₁₆O₁₈S₅ (M+H)⁺: 1559.374, found: 1559.374. Anal. Calcd. for C₆₈H₇₀N₁₆O₁₆S₅2HCl3H₂O: C, 48.42; H, 4.66; N, 13.29; S, 9.50; Cl, 4.28. Found: C, 48.40; H, 4.63; N, 13.18; S, 9.36; Cl, 4.28.

### Example 4

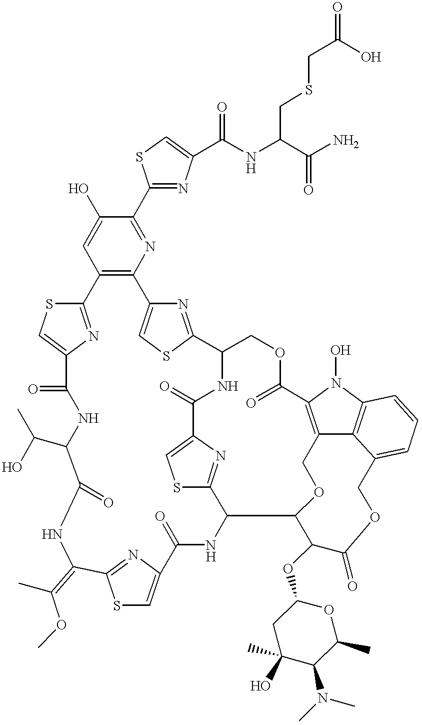

To a stirred suspension of Nocathiacin I (287.5 mg, 0.2 mmol) in water (10 mL) was added triethylamine (0.35 mL, 2.5 mmol). The mixture was stirred until it became a clear yellow solution. To this solution was added mercaptoacetic acid (184 mg, 2 mmol) followed by additional triethylamine (0.28 mL, 2.0 mmol). The reaction mixture was stirred for 3 min and the resulting suspension was stored at −20° C. for 15 h. The reaction mixture was then warmed to room temperature, diluted with saturated sodium bicarbonate (3 mL) and methanol (2 mL) and purified by reverse phase column chromatography using 10-50% methanol/water to give pure product (196 mg, 64%) as a yellow powder. ¹H NMR (DMSO, 500 MHz): δ 10. 82 (bs, 1H), 9.37 (bs, 1H), 9.11 (s, 1H), 8.62 (s, 1H), 8.59 (d, J=8.2 Hz, 1H), 8.52 (s, 1H), 8.43 (s, 1H), 8.26 (s, 1H), 7.89-7.84 (m, 3H), 7.74 (d, J=8.4 Hz, 1H), 7.59 (d, J=7.2 Hz, 1H), 7.36 (m, 2H), 7.22 (d, J=4.4 Hz, 1H), 7.17 (d, J=7.0 Hz, 1H), 5.00 (d, J=12.1 Hz, 1H), 5.74 (m, 1H), 5.68 (d, J=8.6 Hz, 1H), 5.22 (d, J=7.4 Hz, 1H), 5.07 (m, 1H), 5.03 (d, J=12.5 Hz, 1H), 4.93 (d, J=4.6 Hz, 1H), 4.76 (d, J=10.2 Hz, 1H), 4.60 (m, 1H), 4.54 (d, J=11.1 Hz, 1H), 4.31 (d, J=9.6 Hz, 1H), 4.26 (m, 1H), 4.13 (d, J=10.4 Hz, 1H), 4.02 (d, J=9.6 Hz, 1H), 3.93-3.89 (m, 4H), 3.75 (d, J=8.5 Hz, 1H), 3.27-3.17 (m, 6H), 3.12-3.09 (m, 1H), 3.02-2.98 (m, 1H), 2.89 (m, 3H), 2.54 (m, 1H), 2.04 (s, 1H), 1.99 (s, 3H), 1.95 (m, 1H), 1.78 (d, J=14.0 Hz, 1H), 1.41 (s, 3H), 1.16 (m, 2H), 1.10 (m, 4H), 0.56 (d, J=6.6 Hz, 3H). MS: 1529.4 (M+H)⁺, 1527.5 (M−H)⁻; HRMS (ES) calcd. for C₆₃H₆₅N₁₄O₂₀S₆ (M+H)⁺: 1529.282, found: 1529.282. Anal. Calcd. for C₆₃H₆₄N₁₄O₂₀S₆Na.9H₂O: C, 46.14; H, 4.47; N, 11.96; S, 11.73. Found: C, 46.17; H, 4.49; N, 11.80; S, 11.15.

### Example 5

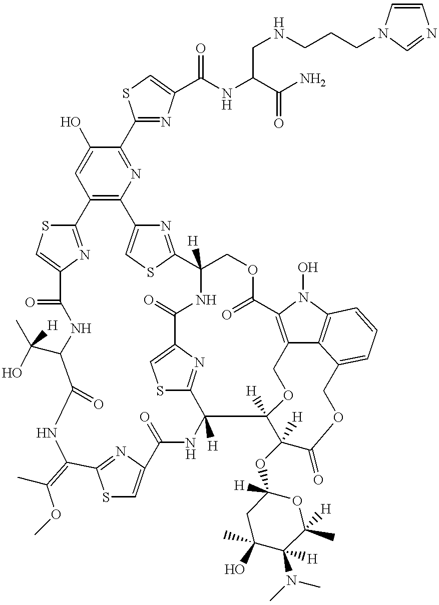

A suspension of Nocathiacin I (0.29 g, 0.2 mmol) in deionized water was treated with 1-(3-aminopropyl)imidazole (0.24 mL, 2.0 mmol). The solution was stirred for 3-4 minutes then stored at −20° C. for 16 hours. The reaction was diluted with water (total volume 40 mL) and quenched by addition of TFA (to pH 3.1). The product was purified by chromatography on C-18, using 3:10 Acetonitrile/H₂O+0.1% TFA as eluent. The lyophilized product was dissolved in H₂O and passed through chloride ion exchange resin to afford the product (0.195 g, 0.125 mmol, 63%) as a yellow lyophilized solid: ¹H NMR (DMSO-d₆, 500 MHz) δ 10.98 (d, J=10.5, 1H), 10.77 (bs, 1H), 9.60 (bs, 1H), 9.42 (m, 1H), 9.13 (s, 1H), 8.78 (bs, 1H), 8.66 (s, 1H), 8.59 (s, 2H), 8.54 (m, 1H), 8.24 (s, 1H), 8.06 (s, 1H), 7.92-7.82 (m, 3H), 7.74 (d, J=8.4, 1H), 7.53 (bs, 1H), 7.35 (m, 2H), 7.19 (d, J=7.0, 1H), 6.40 (bs, 1H), 6.02 (d, J=12.2, 1H), 5.76 (q, J=11.0, 4.0, 1H), 5.72 (d, J=9.2, 1H), 5.22 (m, 1H), 5.06 (m, 3H), 4.96 (m, 1H), 4.79 (d, J=10.4, 1H), 4.52 (d, J=11.3, 1H), 4.30 (d, J=9.6, 1H), 4.25 (t, J=5.6, 1H), 4.15 (d, J=10.7, 1H), 4.05 (d, J=6.6, 1H), 3.91 (s, 3H), 3.89 (m, 1H), 3.67 (m, 1H), 3.51 (t, J=10, 1H), 3.12 (bs 1H), 2.9-2.7 (m, 12H), 2.4 (m, 1H), 2.13 (m, 1H), 2.00 (s, 2H), 1.94 (d, J=14.7, 1H), 1.61 (s, 3H), 1.15 (bs, 3H), 0.80 (d, J=6.8, 3H); MS: 1562.2 (M+H)⁺, 1560.2 (M−H)⁻; Anal. Calcd for C₆₇H₇₁N₁₇NO₈S₅.3.3 HCl.6.7 H₂O: C, 44.61 H, 4.90; N, 13.20; S, 8.89; Cl, 6.49. Found: C, 44.63; H, 4.93; N, 13.06; S, 9.00; Cl, 6.59.

### Example 6

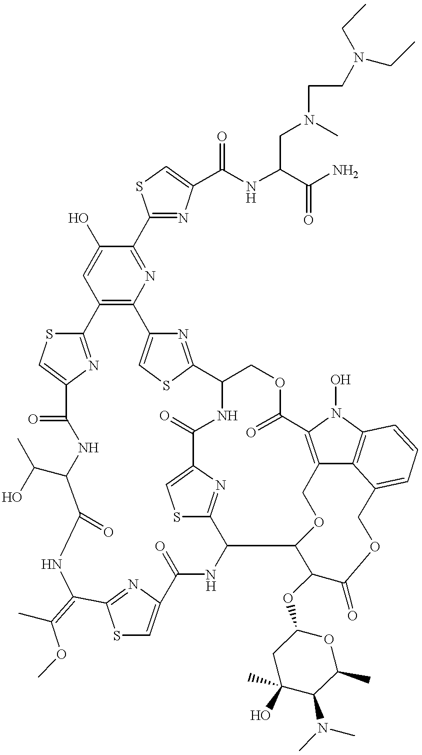

Following the procedure as described for Example 5 except using N,N-Diethyl-N′-(methyl)ethylenediamine (0.32 mL, 2.0 mmol) as the amine component, to afford the product (0.140 g, 0.089 mmol, 45%) as a yellow lyophilized solid: ¹H NMR (DMSO-d₆, 300 MHz) δ 10.95 (m, 1H), 10.79 (bs, 1H), 9.40 (bs, 1H), 9.12 (s, 1H), 8.78 (bs, 1H), 8.64 (s, 1H), 8.57 (m, 2H), 8.52 (s, 1H), 8.22 (s, 1H), 8.03 (s, 1H), 7.92-7.82 (m, 2H), 7.73 (d, J=8.4, 1H), 7.52 (bs, 1H), 7.34 (m, 2H), 7.17 (d, J=7.0, 1H), 6.39 (bs, 1H), 6.01 (d, J=11.8, 1H), 5.74 (m, 1H), 5.70 (d, J=9.5, 1H), 5.20 (m, 1H), 5.03 (m, 3H), 4.78 (d, J=9.5, 1H), 4.51 (d, J=11.0, 1H), 4.29 (d, J=9.5, 1H), 4.25 (t, J=6.1, 1H), 4.13 (d, J=10.3, 1H), 4.05 (d, J=8.7, 1H), 3.90 (s, 3H), 3.89 (m, 1H), 3.70 (m, 1H), 3.12 (m, 4H), 2.9-2.7 (m, 10H), 2.4 (m, 1H), 2.10 (m, 1H), 1.98 (s, 3H), 1.93 (m, 1H), 1.59 (s, 3H), 1.25-1.0 (m, 9H), 0.78 (d, J=6.7, 3H); Anal. Calcd for C₆₈H₇₈N₁₆O₁₈S₅.3.25 HCl.6.5 H₂O: C, 45.29; H, 5.27; N, 12.43; S, 8.89; Cl, 6.39. Found: C, 45.44; H, 5.17; N, 12.46; S, 8.42; Cl, 6.33.

### Example 7

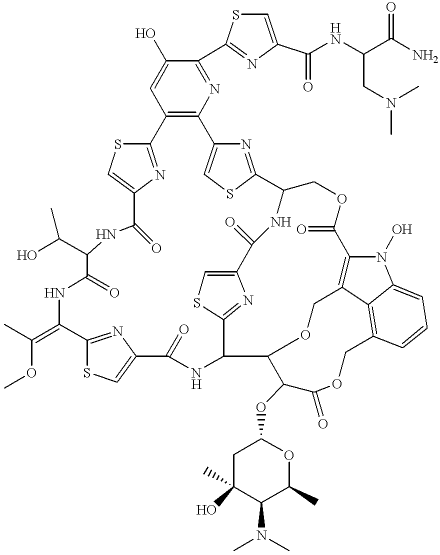

Following the procedure as described for Example 5 except using N,N-Dimethylamine (0.25 mL, 2.0 mmol) as the amine component, to afford the product (0.160 g, 0.108 mmol, 53%) as a yellow lyophilized solid: ¹H NMR (DMSO-d₆, 500 MHz) δ 10.94 (d, J=9.5, 1H), 10.77 (bs, 1H), 9.39 (m, 2H), 9.12 (s, 1H), 8.66 (m, 2H), 8.58 (s, 1H), 8.54 (s, 1H), 8.23 (s, 1H), 8.05 (d, J=2.5, 1H), 7.93-7.80 (m, 3H), 7.75 (d, J=8.2, 1H), 7.54 (bs, 1H), 7.36 (m, 2H), 7.19 (d, J=6.7, 1H), 6.39 (bs, 1H), 6.03 (d, J=12.5, 1H), 5.75 (m, 1H), 5.71 (d, J=8.9, 1H), 5.22 (m, 1H), 5.05 (m, 3H), 4.96 (m, 1H), 4.79 (d, J=10.4, 1H), 4.52 (d, J=10.7, 1H), 4.30 (d, J=9.8, 1H), 4.25 (t, J=6, 1H), 4.15 (d, J=10.7, 1H), 4.04 (d, J=9.2, 1H), 3.91 (s, 3H), 3.88 (m, 1H), 3.66 (m, 1H),3.49 (t, J=11, 1H) 3.12 (bs, 1H), 2.9-2.6 (m, 13H), 2.46 (bs, 1H), 2.12 (m, 1H), 1.99 (s, 3H), 1.94 (d, J=14.7, 1H), 1.60 (s, 3H), 1.16 (bs, 3H), 0.81 (d, J=6.7, 3H); MS: 1482 (M+H)⁺; HRMS (ES) found: 1482.34590; Anal. Calcd for C₆₃H₆₇N₁₅O₁₈S₅2.25 HCl.7.0 H₂O: C, 44.75; H, 4.96; N, 12.43; S, 9.48; Cl, 4.72. Found: C, 44.96; H, 4.87; N, 12.48; S, 9.30; Cl, 6.59.

### Example 8

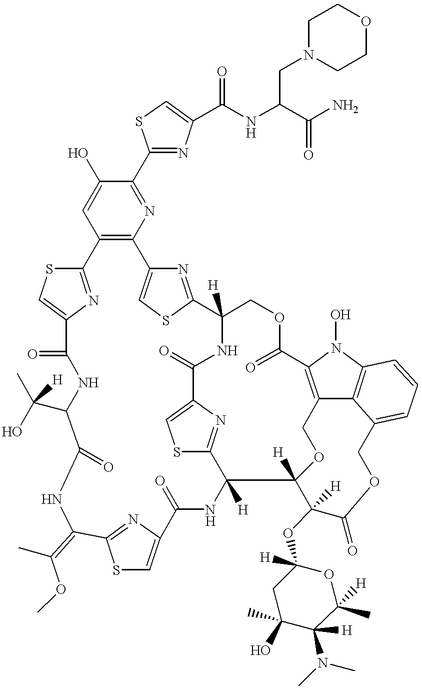

Following the procedure for Example 5 except using nocathiacin I (0.5 mmol, 0.719 g.), and morpholine (0.87 mL, 10.0 mmol) as the amine component, to afford the product (0.345 g, 0.224 mmol, 43%) as a yellow lyophilized solid: ¹H NMR (DMSO-d₆, 500 MHz) δ 10.98 (m, 1 H), 10.79 (bs, 1H), 9.46 (bs, 1H), 9.12 (s, 1H), 8.75 (bs, 1H), 8.65 (s, 1H), 8.58 (m, 2H), 8.54 (s, 1H), 8.22 (s, 1H), 8.05 (s, 1H), 7.92-7.82 (m, 2H), 7.74 (d, J=8.4, 1H), 7.52 (bs, 1H), 7.34 (m, 2H), 7.19 (d, J=7.1, 1H), 6.39 (bs, 1H), 6.02 (d, J=12.1, 1H), 5.75 (m, 1H), 5.71 (d, J=10.0, 1H), 5.23 (m, 1H),5.07-4.90 (m, 3H), 4.78 (d, J=10.3, 1H), 4.62 (d, J=10.7, 1H), 4.30 (d, J=9.6, 1H), 4.25 (t, J=5.5, 1H), 4.14 (d, J=10.5, 1H), 4.05 (d, J=8.7, 1H), 3.97 (m, 1H), 3.91 (s, 3H), 3.78 (m, 2H), 3.22 (bs, 2H), 3.12 (bs, 1H), 2.89 (m, 7H), 2.47 (bs, 1H), 2.13 (m, 1H), 2.00 (s, 3H), 1.93 (d, J=14.7, 1H), 1.61 (s, 3H), 1.15 (bs, 3H), 0.81 (d, J=7.0, 3H); MS: 1524.5 (M+H)⁺, 1522.6 (M−H)⁻; Anal. Calcd for C₆₅H₆₉N₁₅O₁₉S₅.2.5 HCl.5.8 H₂O: C, 45.38; H, 4.87; N, 12.21; S, 9.32; Cl, 5.15. Found: C, 45.11; H, 4.71; N, 12.15; S, 9.25; Cl, 5.19.

### Example 9

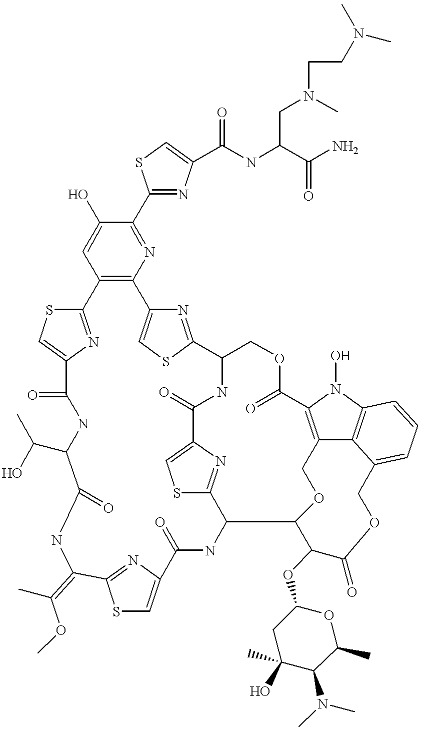

Following the procedure as described for Example 5 except using N,N,N′-Trimethylethylenediamine (0.26 mL, 2.0 mmol) as the amine component, to afford the product (0.135 g, 0.088 mmol, 44%) as a yellow lyophilized solid: ¹H NMR (DMSO-d₆, 300 MHz) δ 10.96 (bs, 1H), 10.79 (bs, 1H), 9.12 (s, 1H), 8.75 (bs, 1H), 8.64 (s, 1H), 8.57 (m, 2H), 8.52 (s, 1H), 8.22 (s, 1H), 8.03 (s, 1H), 7.92-7.82 (m, 2H), 7.73 (d, J=8.5, 1H), 7.34 (m, 2H), 7.17 (d, J=6.7, 1H), 6.38 (bs, 1H), 6.01 (d, J=11.9, 1H), 5.74 (m, 1H), 5.70 (d, J=9.2, 1H), 5.20 (m, 1H), 5.04 (m, 3H), 4.77 (d, J=9.1, 1H), 4.51 (d, J=11.1, 1H), 4.29 (d, J=9.6, 1H), 4.24 (m, 1H), 4.13 (d, J=10.6, 1H), 4.04 (d, J=9.8, 1H), 3.90 (s, 3H), 3.88 (m, 1H), 3.10 (s, 1H), 2.9-2.7 (m, 10H), 2.4 (m, 1H), 2.10 (m, 1H), 1.98 (s, 3H), 1.93 (m, 1H), 1.59 (s, 3H), 1.3 (m, 3H), 0.78 (d, J=7.1, 3H); MS: 1540.4 (M+H)⁺, 1537.5 (M−H)⁻; Anal. Calcd for C₆₆H₇₄N₁₆O₁₈S₅.3.25 HCl.7.4 H₂O: C, 44.25; H, 5.18; N, 12.51; S, 8.95; Cl, 6.43. Found: C, 44.64; H, 4.80; N, 12.64; S, 8.76; Cl, 6.85.

### Example 10

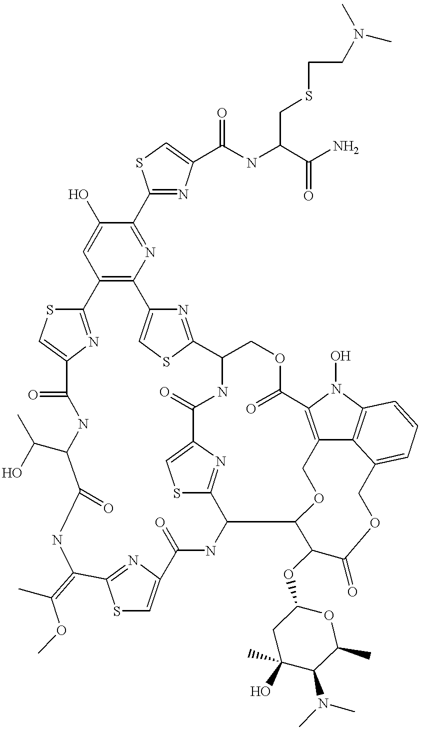

To a stirred suspension of Nocathiacin I (287.5 mg, 0.2 mmol) in water (10 mL) was added triethylamine (0.34 mL, 2.43 mmol) and stirred until the reaction mixture became a clear yellow solution. To this solution was added 2-(N,N-dimethylamino)ethanethiol hydrochloride (284 mg, 2 mmol), stirred for 3 min and left at −20° C. for 2.5 h. The reaction mixture was then warmed to room temperature, diluted with 1 M aqueous HCl (3 mL) and water (20 mL) and purified by reverse phase column chromatography on C-18 using 10-80% methanol/water containing about 0.01% HCl to provide pure product (277 mg, 87%) as a yellow powder. MS: 1542.6 (M+H)⁺, 1540.6 (M−H)⁻; HRMS: found 1542.354.

### Examples 11-56, 58-92

Examples 11-56 and 58-92 were prepared according to either Method A or Method B as described directly below.

Method A, General procedure for the Michael addition of amines to a Nocathiacin

The following general procedure was used to prepare the compounds of examples 11-33, 35-39, 41, 48-59, 61, 63-79 and 81-92 with the exception that the Nocathiacin in which R² is H was used as starting material for example 92. To a stirred solution of an appropriate amine of general formula HNRR¹ (3.50 mmol, 10 equivalents) in water (17.5 mL) at room temperature was added Nocathiacin I (0.5 g, 0.35 mmol, 1 equivalent). The reaction mixture was stirred for 3 to 5 minutes until it became a clear homogeneous solution. If the reaction did not become a clear homogeneous solution then triethylamine (0.70-1.40 mmol, 24 equivalents) was added. The reaction mixture was then maintained at −20° C. for a period of 3 to 20 h. The reaction mixture was then worked up and the product purified by one of the following methods: a) reaction quenched with 1 N aqueous hydrochloric acid and subjected to medium pressure chromatography, as described above; b) reaction quenched with trifluoroacetic acid and subjected to preparative HPLC or medium pressure chromatography on C-18; or c) the reaction mixture directly subjected to medium pressure chromatography or preparative HPLC. Examples 73 and 74 were isolated as single diastereomers (the stereochemistry of the carbon to which —CH₂YR¹ is attached is denoted (R) or (S) in the following table) from the same reaction mixture by preparative HPLC. Examples 75 and 76 were also isolated as single diastereomers by the same method. The product containing fractions were concentrated under reduced pressure, frozen and lyophilized to afford the product as a yellow powder.

Method B: General procedure for the Michael addition of thiols to a Nocathiacin

The following general procedure was used to prepare the compounds of examples 34, 40, 42-47, 60, 62 and 80. To a stirred solution of triethylamine (12.5 equivalents) in water (17.5 mL) at room temperature was added Nocathiacin I (0.5 g, 0.35 mmol, 1 equivalent). To this was added an appropriate thiol of general formula HSR¹ (3.50 mmol, 10 equivalents). The reaction mixture was stirred for 3 to 5 minutes until it became a clear homogeneous solution. If the reaction did not become a clear homogeneous solution then additional triethylamine (0.70-1.40 mmol, 2-4 equivalents) was added. The reaction mixture was then maintained at −20° C. for a period of 3 to 20 h. The reaction mixture could then be purified by procedures analogous to those described for Method A, above.

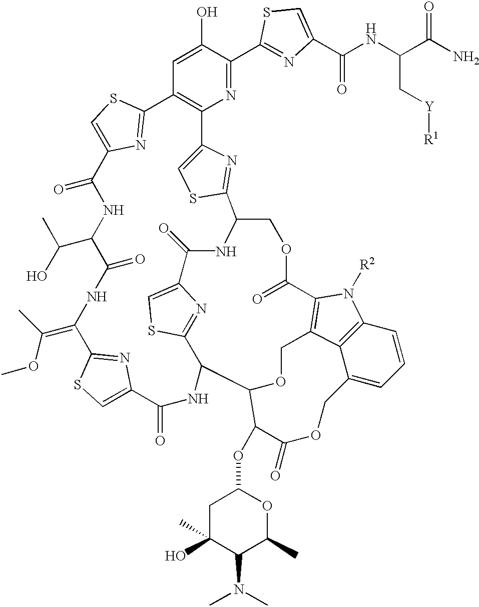

| Example | | | MS observed | MS observed | HRMS |
|---|---|---|---|---|---|
| Number | R² | Y-R¹ | (M + H)⁺ | (M − H)⁻ | observed |
| 1 | OH | 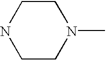 | 1538.1 | 1535.54 | 1537.390 |
| 2 | OH | 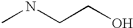 | 1512.4 | 1511.60 | 1512.359 |
| 3 | OH | 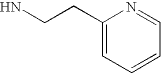 | 1559.7 | 1558.46 | 1559.374 |
| 4 | OH | 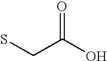 | 1529.4 | 1527.47 | 1529.282 |
| 5 | OH | 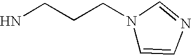 | 1562.2 | 1560.17 | |
| 6 | OH | 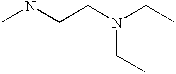 | 1567.5 | 1565.6 | |
| 7 | OH | N(CH₃)₂ | 1482 | | 1482.34590 |
| 8 | OH | 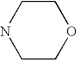 | 1524.5 | 1522.59 | |
| 9 | OH | 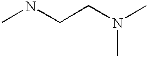 | 1540.4 | 1537.5 | |
| 10 | OH | 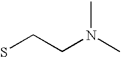 | 1542.6 | 1540.65 | 1542.354 |
| 11 | OH | 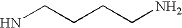 | 1525 | | 1525.39048 |
| 12 | OH | 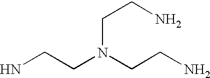 | 1583 | | 1583.44636 |
| 13 | OH | NHCH₃ | 1468.6 | 1466.53 | |
| 14 | OH | 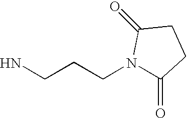 | 1579.1 | 1578.18 | |
| 15 | OH | 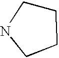 | 1508.4 | 1507.65 | 1508.364 |
| 16 | OH | 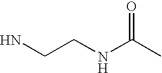 | 1539.2 | 1537.4 | 1539.366 |
| 17 | OH | 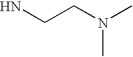 | 1525.7 | 1523.2 | |
| 18 | OH | NHOH | 1471.3 | 1468.9 | |
| 19 | OH | 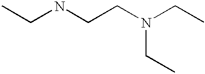 | 1567.5 | 1565.6 | |
| 20 | OH | 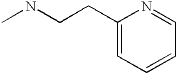 | 1573.5 | 1571.60 | 1573.387 |
| 21 | OH | 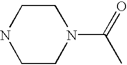 | 1565.5 | 1563.86 | 1565.386 |
| 22 | OH | 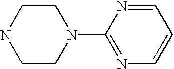 | 1601.6 | 1600.86 | 1601.401 |
| 23 | OH | 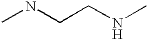 | 1525.5 | 1524.3 | |
| 24 | OH | 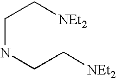 | 1652.7 | 1650.8 | |
| 25 | OH | 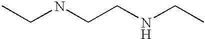 | 1553.6 | | |
| 26 | OH | 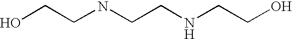 | 1585.7 | 1584.1 | |
| 27 | OH | 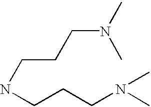 | 1624.6 | 1623.2 | |
| 28 | OH | 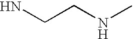 | 1511.5 | 1509.7 | |
| 29 | OH | 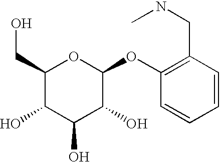 | 1736.1 | | 1736.429 |
| 30 | OH | 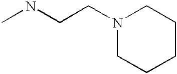 | 1579.8 | 1579.00 | 1579.436 |
| 31 | OH | 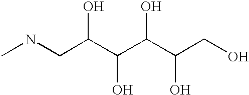 | 1632.7 | 1630.71 | 1632.399 |
| 32 | OH | 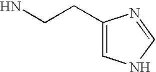 | 1548.7 | 1546.84 | 1548.367 |
| 33 | OH | 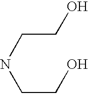 | 1542.2 | | 1542.367 |
| 34 | OH | 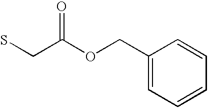 | 1619.1 | | 1619.331 |
| 35 | OH | 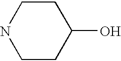 | 1539.8 | 1537.68 | 1538.375 |
| 36 | OH | 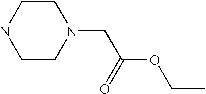 | 1609.7 | 1608.23 | 1609.412 |
| 37 | OH | 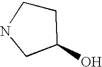 | 1525.7 | 1523.68 | 1524.355 |
| 38 | OH | HO—N—CH₃ | 1484.2 | | |
| 39 | OH | 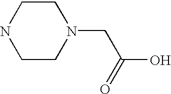 | 1581.7 | 1579.73 | 1581.375 |
| 40 | OH | 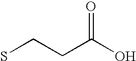 | 1543.5 | 1541.62 | 1543.302 |
| 41 | OH | 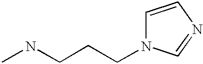 | 1576.8 | 1574.85 | 1576.402 |
| 42 | OH | 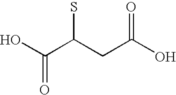 | 1587.4 | 1585.51 | 1587.284 |
| 43 | OH | 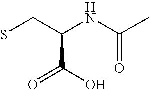 | 1600.5 | 1598.53 | 1600.317 |
| 44 | OH | 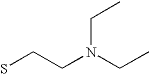 | 1570.6 | 1568.67 | 1570.386 |
| 45 | OH | 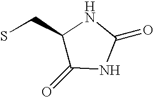 | 1583.7 | 1581.70 | 1583.301 |
| 46 | OH | 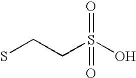 | 1579.5 | 1577.62 | 1579.266 |
| 47 | OH | 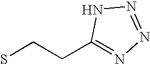 | 1567.4 | 1565.59 | 1567.319 |
| 48 | OH | 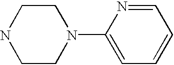 | 1600.7 | 1598.74 | 1600.404 |
| 49 | OH | 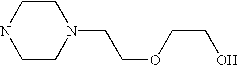 | 1611.3 | | 1611.422 |
| 50 | OH | 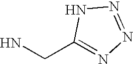 | 1536.3 | | 1536.345 |
| 51 | OH | 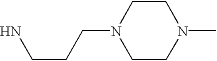 | 1594.5 | | 1594.443 |
| 52 | OH | 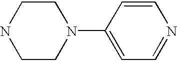 | 1600.8 | | 1600.400 |
| 53 | OH | 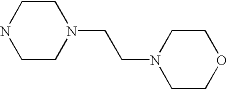 | 1636.9 | 1634.96 | 1636.455 |
| 54 | OH | 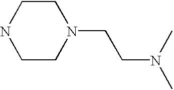 | 1596 | | |
| 55 | OH | 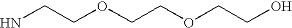 | 1586.8 | 1584.86 | 1586.393 |
| 56 | OH | 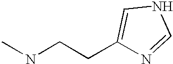 | 1562.7 | 1560.79 | |
| 58 | OH | 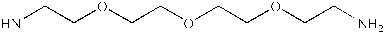 | 1629.6 | 1627.74 | 1629.437 |
| 59 | OH | 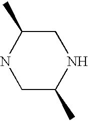 | 1551.8 | 1549.89 | 1551.404 |
| 60 | OH | 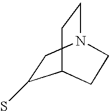 | 1580.6 | 1578.67 | 1580.363 |
| 61 | OH | 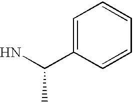 | 1558.3 | | 1558.374 |
| 62 | OH | 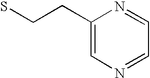 | 1577.6 | 1575.71 | 1577.329 |
| 63 | OH | 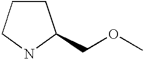 | 1552.6 | 1550.76 | 1552.393 |
| 64 | OH | 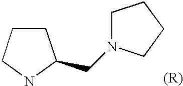 | 1591.5 | | 1591.434 |
| 65 | OH | 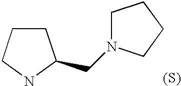 | 1591.5 | | 1591.431 |
| 66 | OH | 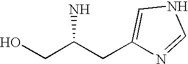 | 1578.4 | 1576.74 | 1578.378 |
| 67 | OH | 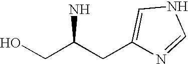 | 1579.8 | 1576.63 | 1578.383 |
| 68 | OH | 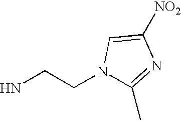 | 1607.6 | 1605.54 | 1607.365 |
| 69 | OH | [chemical structure] | 1713.5 | | 1713.496 |
| 70 | OH | 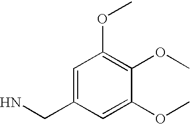 | 1635 | | 1634.392 |
| 71 | OH | 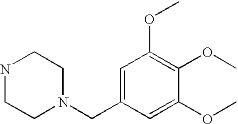 | 1704 | | 1703.447 |
| 72 | OH | 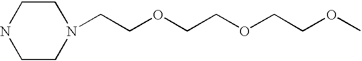 | 1669.8 | 1667.9 | 1669.463 |
| 73 | OH | 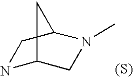 | 1549.7 | 1547.68 | 1549.391 |
| 74 | OH | 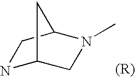 | 1549.6 | 1547.71 | 1549.385 |
| 75 | OH | 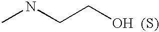 | 1513.1 | 1510.76 | |
| 76 | OH | 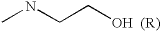 | 1512.6 | 1510.65 | |
| 77 | OH | 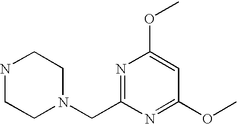 | 1675.5 | | 1675.429 |
| 78 | OH | 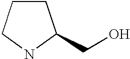 | 1538.6 | 1536.56 | 1538.374 |
| 79 | OH | 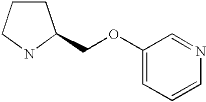 | 1616.1 | 1613.65 | 1615.404 |
| 80 | OH | 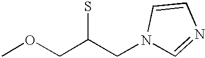 | 1609.6 | 1607.56 | 1609.355 |
| 81 | OH | 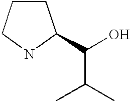 | 1580.6 | 1578.84 | 1580.418 |
| 82 | OH | 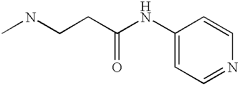 | 1617.3 | 1614.64 | |
| 83 | OH | 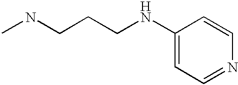 | 1604.1 | 1601.98 | |
| 84 | OH | 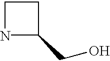 | 1524.7 | 1523.96 | |
| 85 | OH | 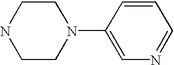 | 1600.3 | | 1600.400 |
| 86 | OH | 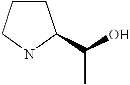 | 1553.7 | 1550.92 | 1552.391 |
| 87 | OH | 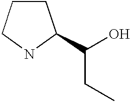 | 1566.3 | | |
| 88 | OH | 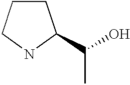 | 1552.6 | 1550.74 | 1552.387 |
| 89 | OH | 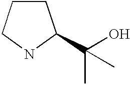 | 1566.6 | 1565.62 | 1566.405 |
| 90 | OH | 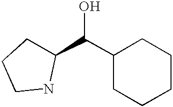 | 1620.7 | 1619.78 | 1620.452 |
| 91 | OH | 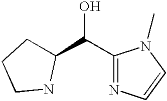 | 1619.7 | 1617.75 | 1618.409 |
| 92 | H | 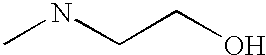 | 1494.4 | 1496.7 | |

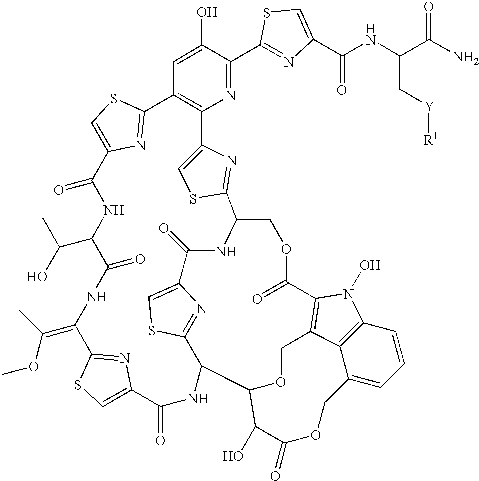

| | | | | HRMS if |
|---|---|---|---|---|
| Example | Y-R¹ | (M + H)⁺ | (M − H)⁻ | available |
| 93 | 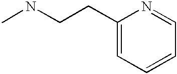 | 1402.3 | 1401.29 | 1402.2667 |
| 94 | 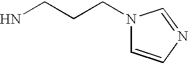 | 1391.4 | 1389.6 | 1391.262 |
| 95 | 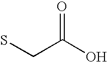 | 1358.2 | 1356.3 | 1358.159 |
| 96 | 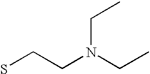 | 1355.3 | 1353.2 | 1355.247 |
| 97 | 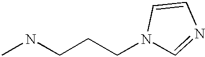 | 1405.6 | 1403.49 | 1405.278 |
| 98 | 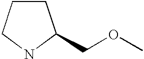 | 1382.5 | 1379.43 | |

### Example 99

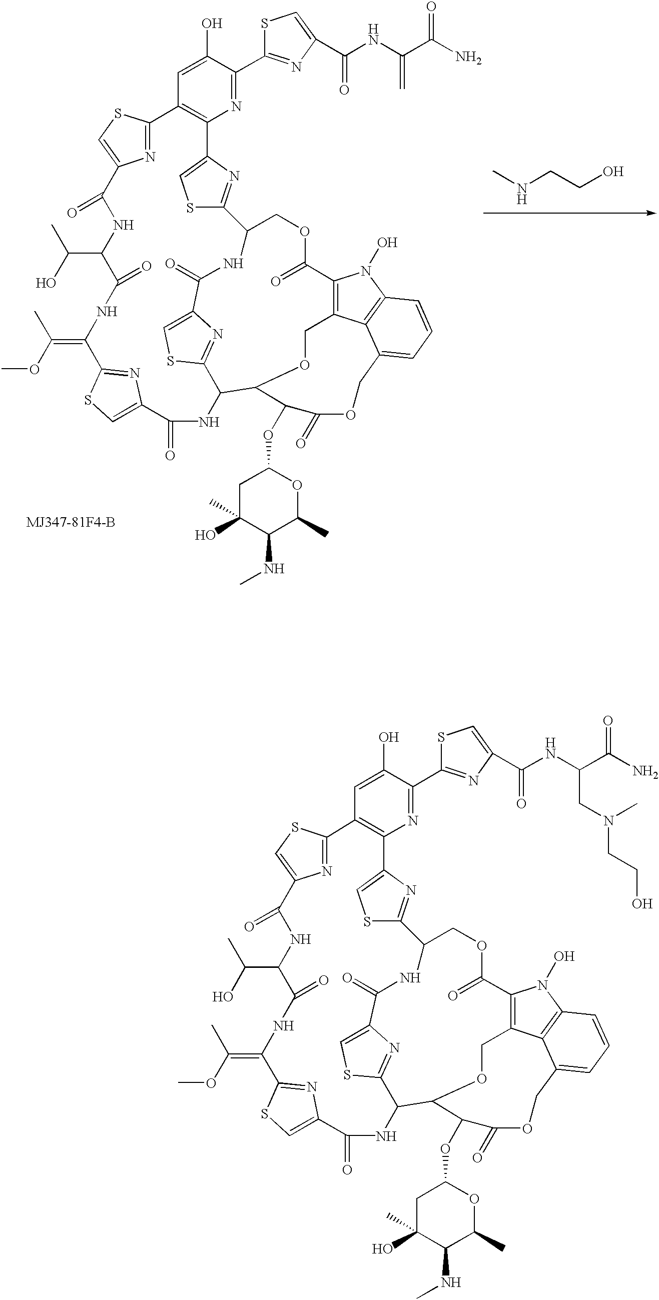

To a stirred solution of 2-(Methylamino)ethanol (11 mg, 0.15 mmol, 10 equivalents) in water (1.0 mL) at room temperature was added the compound MJ347-81F4-B (20.5 mg, 1 equivalent). The reaction mixture was stirred for 3 to 5 minutes until it became a clear homogeneous solution then was maintained at −20° C. for 16 h. The reaction mixture was then subjected to preparative HPLC. The product fraction was concentrated in vacuo, frozen and lyophilized to provide the bis trifluoroacetic acid salt of the product as a yellow solid. Calcd. Mass for C₆₃H₆₇N₁₅O₁₉S₅ is 1497.334. Found MS: 1498.4 (M+H)⁺, 1496.61 (M−H)⁻; HRMS: 1498.344.

### Example 100

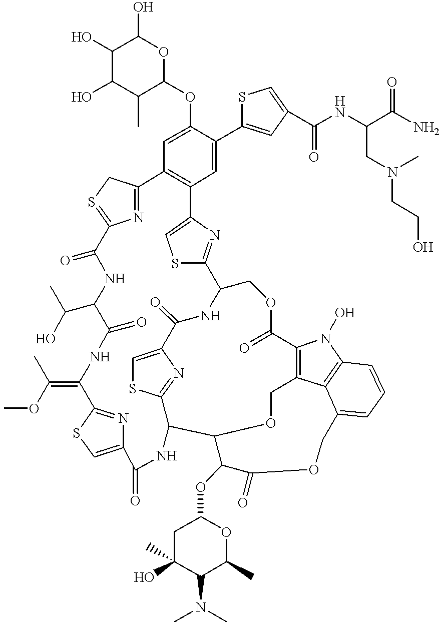

To a suspension of the appropriate nocathiacin derivative (52 mg) in water (5 mL) was added 2-(Methylamino)ethanol. The mixture was stirred for 5 minutes at room temperature and became a yellowish green solution. The mixture was then maintained at −20° C. for 20 h. The mixture was then purified by reverse phase column chromatography on C-18 using 10-80% acetonitrile/water containing about 0.01% HCl to provide the bis hydrochloride salt of the product as a yellow solid. Calcd. Mass for C₇₀H₇₉N₁₅O₂₃S₅ is 1657.82. Found MS: 1658.9 (M+H)⁺, 1657.18 (M−H)⁻.

### Example 101

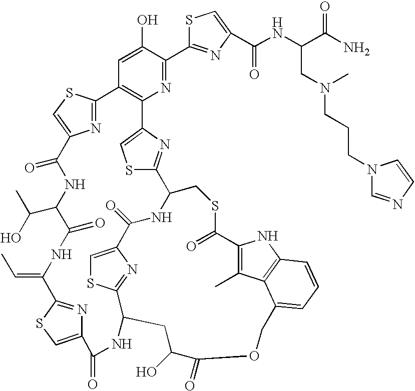

To a stirred solution of nosiheptide (69 mg, 0.056 mmol) in water (3 mL) and DMF (1 mL) was added 1-(3-N-methylaminopropyl)imidazole (140 mg, 1 mmol) and the resulting dark solution was left for 3 days at −20° C. The reaction mixture was then allowed to warm to room temperature, diluted with methanol and purified by preparative HPLC to provide the bis trifluoroacetic acid salt of the product (4.0 mg, 5%) as a yellow powder. Calcd. Mass for C₅₈H₅₇N₁₆O₁₂S₆ is 1361.267. Found MS: 1361.9 (M+H)⁺, 1359.4 (M−H)⁻; HRMS: Found 1361.269 (M+H)⁺.

### Example 102

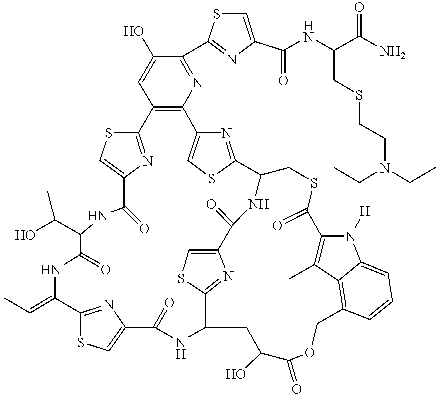

To a stirred suspension of nosiheptide (61.12 mg, 0.05 mmol) and 2-(N,N-diethylamino)ethanethiol hydrochloride (42.43 mg, 0.25 mmol) in water (5 mL) was added triethylamine (70 μL, 0.5 mmol) and stirred for 15 minutes at room temperature. To the resulting suspension DMF (3 mL) was added, stirred for 5 minutes and the clear yellow solution was left at −20° C. for 20 h. The reaction mixture was then allowed to warm to room temperature, diluted with methanol and purified by preparative HPLC to provide the bis trifluoroacetic acid salt of the product as a yellow powder. ¹H NMR (500 MHz, DMSO-d₆): 10.85 (d, J=4.2 Hz,1H), 9.41 (bs, 1H), 9.24 (d, J=8.7 Hz, 1H), 9.05 (bs, 1H), 8.69 (bs, 1H), 8.55 (s, 1H), 8.44 (bs, 1H), 8.31 (s, 1H), 8.19 (s, 1H), 7.93 (t, J=3.1 Hz, 1H), 7.90 (s, 1H), 7.78 (d, J=9.4 Hz, 1H), 7.65 (s, 1H), 7.54 (bs, 1H), 7.33 (s, 1H), 7.28 (t, J=7.5 Hz, 1H), 7.14 (d, J=7.0 Hz, 1H), 6.47 (q, J=6.8, 13.7 Hz, 1H), 5.86 (bs, 1H), 5.71 (bs, 1H), 5.58-5.55 (m, 2H), 4.68 (m, 2H), 4.58 (bs, 1H), 4.08 (d, J=12.3 Hz, 2H), 3.85-3.81 (m, 2H), 3.53 (bs, 2H), 3.28-3.13 (m, 9H), 2.98-2.88 (m, 4H), 2.71-2.55 (m, 2H), 2.54 (s, 1H), 1.72 (d, J=6.75 Hz, 4H), 1.20-1.16 (m, 8H); Calcd. (M+H)⁺ (ESI) for C₅₇H₅₈N₁₄O₁₂S₇: 1355.248, found: 1355.247.

### Example 103

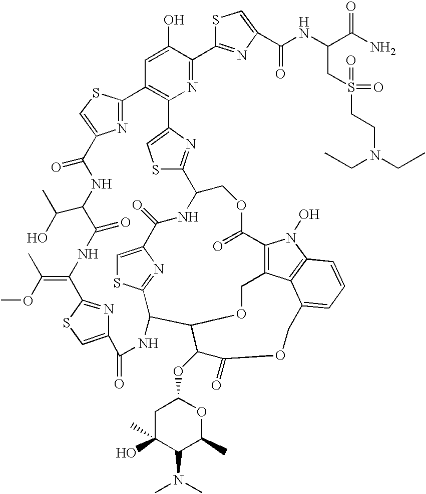

To a stirred solution of the compound of example 44 (as the hydrochloride salt, 336 mg, 0.19 mmol) in water (40 mL) was added sodium tungstate dihydrate (15 mg, 0.045 mmol) followed by 30% aqueous hydrogen peroxide (1 mL) at room temperature. After 3 h, the reaction mixture was purified by reverse phase column chromatography on C-18 using 10-80% methanol/water containing about 0.01% HCl to provide pure product (232 mg, 68%) as a yellow powder. MS: 1603.0 (M+H)⁺, 1600.6 (M−H)⁻; HRMS: found 1602.372.

### Example 104

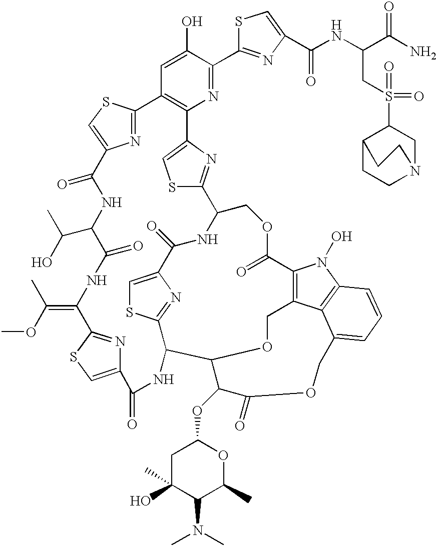

Example 104 was prepared by the same method as described for Example 103 starting from the compound of Example 60 (as the hydrochloride salt) to provide the product as a yellow powder. MS: 1613.5 (M+H)⁺, 1611.5 (M−H)⁻; HRMS: found 1612.354.

### Example 105

[chemical structure]

To a solution of the compound of example 60 (as the free base, 116.0 mg, 0.07 mmol) in water (10 mL) was added sodium tungstate dihydrate (5 mg, 0.015 mmol) followed by 30% hydrogen peroxide (0.5 mL). The reaction mixture was stirred at room temperature 3 hours then was purified by reverse phase column chromatography on C-18 using 10-80% methanol/water containing about 0.01% HCl to provide pure product as a yellow powder. MS: (M+H)⁺1645.9, (M−H)⁻1642.80; HRMS: found 1644.344; Anal. Calcd for C₆₈H₇₃N₁₅O₂₂S₆.2.0 HCl.10.0 H₂O: C, 43.04; H, 5.05; N. 11.07; S, 10.14; Cl, 3.74. Found: C, 42.50; H, 4.89; N, 10.80; S, 9.92; Cl, 3.73.

### Examples 106-111

Examples 106-109 were prepared according to the following general procedure for the preparation of amides and ureas of Michael adducts:

To a stirred solution of appropriate amine (10 equiv.) in water at room temperature was added Nocathiacin I (1 equiv.). The reaction mixture was stirred for 3-5 minutes until it became a clear homogeneous solution. Then the reaction mixture was maintained at −20° C. for 16 h. An appropriate acid anhydride or isocyanate ( 10 equiv.) was then added to the reaction mixture and the mixture was allowed to warm to room temperature. Once the reaction was done as judged by analytical HPLC analysis, it was purified by preparative HPLC to afford the compounds of examples 106-109 (as shown in the table below) as a yellow powder. For examples 106-109, methylamine (40 weight % solution in water) was employed as the amine component. For examples 106 and 107 the anhydride component employed was succinic anhydride and acetic anhydride, respectively. Examples 108 and 109 were obtained from the reaction employing methyl isocyanate as the isocyanate component.

Examples 110 and 111 were prepared according to the following procedure:

To a solution of the compound of example 17 (77 mg, 0.05 mmol) in pyridine (1 mL) and DMF (0.5 mL) at room temperature was added methyl isocyanate (3 μL, 0.05 mmol). The reaction mixture was stirred at room temperature for approximately 1 hour then was concentrated in vacuo. The residue was dissolved in DMF (1.7 mL) and was purified by preparative HPLC according to the general method to provide two product components. The earlier eluting component was found to be the compound designated example 111 and the later eluting component was found to be the compound designated example 110.

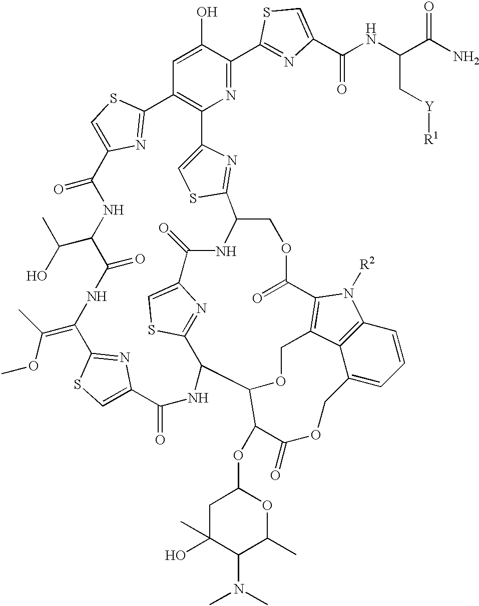

| | | | | | HRMS if |
|---|---|---|---|---|---|
| Example | R² | Y-R¹ | (M + H)⁺ | (M − H)⁻ | available |
| 106 | OH | 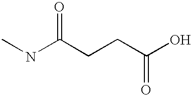 | 1568.3 | | 1568.348 |
| 107 | CH₃C(O)O | CH₃CONCH₃ | 1552.6 | 1551.7 | 1552.350 |
| 108 | 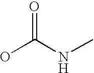 | 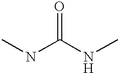 | 1582.4 | 1580.52 | 1582.3752 |
| 109 | OH | 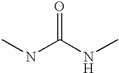 | 1525.3 | | 1525.357 |
| 110 | OH | 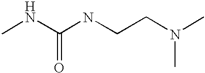 | 1582.4 | 1579.9 | |
| 111 | 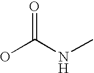 | 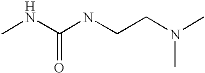 | 1639.3 | 1638.0 | |

Biological Activity

In vitro Antibiotic Activity of Formula I Compounds

To demonstrate its antimicrobial properties, the minimum inhibitory concentration (MIC) for compounds of the invention was obtained against a variety of bacteria using a conventional broth micro dilution assay in accordance with standards recommended by the National Committee for Clinical Laboratory Standards (NCCLS). The serial broth dilution method used Mueller-Hinton medium except for the Streptococcus pneumoniae which was tested in 50% Mueller-Hinton medium and 50% Todd Hewitt medium. The final bacterial inoculate contained approximately 5×10⁵ cfu/well and was run on microtiter plates. The volume of each well was 100 μL and the plates were incubated at 35° C. for 18 hours in ambient air. The MIC was defined as the lowest drug concentration that prevented visible growth. Some of the results obtained are shown in Table 1 below, and demonstrate that compounds of this invention have utility in treating bacterial infections. Column 1 of Table 1 provides the compound example number and columns 2, 3, and 4 provide the MIC obtained by that compound against the specified strain of organism.

TABLE 1

| | MIC (μg/mL) | MIC (μg/mL) | MIC (μg/mL) |
|---|---|---|---|
| | against | against | against |
| | Staphylococcus | Streptococcus | Enterococcus |
| EXAMPLE | aureus | pneumoniae | faecalis |
| number | A15090 | A28272 | A20688 |
| 1 | 0.25 | 0.125 | 0.5 |
| 2 | 0.015 | 0.015 | 0.06 |
| 3 | 1.0 | 0.25 | 1.0 |
| 4 | 2.0 | 0.125 | 2.0 |
| 5 | 1.0 | 0.03 | 1.0 |
| 6 | 0.125 | 0.03 | 2.0 |
| 7 | 0.06 | 0.03 | 0.125 |
| 8 | 0.015 | 0.003 | 0.125 |
| 9 | 0.06 | 0.007 | 0.5 |
| 10 | 0.125 | 0.015 | 0.25 |
| 11 | 2.0 | 0.5 | 8.0 |
| 12 | 2.0 | 0.5 | 4.0 |
| 13 | 2.0 | 0.001 | 8.0 |
| 14 | 1.0 | 0.06 | 1.0 |
| 15 | 0.06 | 0.015 | 0.5 |
| 16 | 1.0 | 0.125 | 4.0 |
| 17 | 2.0 | 0.03 | 4.0 |
| 18 | 0.5 | 0.03 | 4.0 |
| 19 | 0.25 | 0.03 | 1.0 |
| 20 | 0.125 | 0.005 | 0.25 |
| 21 | 0.5 | 0.03 | 2.0 |
| 22 | 0.5 | 0.0005 | 1.0 |
| 23 | 0.06 | 0.001 | 0.5 |
| 24 | 1.0 | 0.007 | 2.0 |
| 25 | 1.0 | 0.0005 | 2.0 |
| 26 | 1.0 | 0.001 | 2.0 |
| 27 | 4.0 | 0.0005 | 2.0 |
| 28 | 0.25 | 0.03 | 0.5 |
| 29 | 0.06 | 0.015 | 0.25 |
| 30 | 0.125 | 0.03 | 1.0 |
| 31 | 0.125 | 0.125 | 1.0 |
| 32 | 0.25 | 0.125 | 4.0 |
| 33 | 0.125 | 0.015 | 0.25 |
| 34 | 1.0 | 0.001 | 1.0 |
| 35 | 0.06 | 0.007 | 0.25 |
| 36 | 0.06 | 0.007 | 0.25 |
| 37 | 0.06 | 0.007 | 0.125 |
| 38 | 0.03 | 0.007 | 0.06 |
| 39 | 1.0 | 0.03 | 1.0 |
| 40 | 1.0 | 0.03 | (4.0) |
| 41 | 0.5 | 0.125 | 0.5 |
| 42 | 4.0 | 0.25 | 4.0 |
| 43 | 2.0 | 0.125 | 8.0 |
| 44 | 0.125 | 0.015 | 0.25 |
| 45 | 4.0 | 0.03 | 128 |
| 46 | 4.0 | 0.06 | 128 |
| 47 | 2.0 | 0.25 | 32 |
| 48 | 0.125 | 0.03 | 0.25 |
| 49 | 0.5 | 0.5 | 2.0 |
| 50 | 0.25 | 0.25 | 1.0 |
| 51 | 0.25 | 0.015 | 4.0 |
| 52 | 0.5 | 0.5 | 1.0 |
| 53 | 0.5 | 0.5 | 1.0 |
| 54 | 0.5 | 0.25 | 0.125 |
| 55 | 0.25 | 0.06 | 0.5 |
| 56 | 0.25 | 0.125 | 0.5 |
| 58 | 1.0 | 0.25 | 1.0 |
| 59 | 1.0 | 0.25 | 2.0 |
| 60 | 2.0 | 0.5 | 2.0 |
| 61 | 0.25 | 0.03 | 0.25 |
| 62 | 0.125 | 0.015 | 0.25 |
| 63 | 0.125 | 0.03 | 0.25 |
| 64 | 0.06 | 0.003 | 1.0 |
| 65 | 0.03 | 2.0 | 1.0 |
| 66 | 2.0 | 2.0 | 16 |
| 67 | 2.0 | 1.0 | 8.0 |
| 68 | 2.0 | 0.5 | 8.0 |
| 69 | 0.5 | 0.25 | 4.0 |
| 70 | 0.25 | 0.06 | 4.0 |
| 71 | 1.0 | 0.5 | 2.0 |
| 72 | 4.0 | 0.5 | 4.0 |
| 73 | 0.25 | 0.015 | 2.0 |
| 74 | 0.5 | 0.015 | 2.0 |
| 75 | 0.06 | 0.007 | 0.25 |
| 76 | 0.06 | 0.007 | 0.25 |
| 77 | 0.125 | 0.015 | 0.5 |
| 78 | 0.03 | 0.007 | 0.125 |
| 79 | 0.06 | 0.001 | 0.125 |
| 80 | 0.25 | 0.03 | 0.125 |
| 81 | 0.125 | 0.007 | 0.125 |
| 82 | 0.5 | 0.03 | 0.5 |
| 83 | 1.0 | 0.25 | 0.5 |
| 84 | 0.5 | 0.125 | 0.25 |
| 85 | 0.06 | 0.007 | 0.25 |
| 86 | 0.007 | 0.015 | 0.06 |
| 87 | 0.015 | 0.007 | 0.06 |
| 88 | 0.007 | 0.03 | 0.06 |
| 89 | 0.003 | 0.007 | 0.03 |
| 90 | 0.03 | 0.007 | 0.06 |
| 91 | 0.03 | 0.015 | 0.06 |
| 92 | 0.125 | 0.0005 | 0.125 |
| 93 | 0.06 | 0.001 | 0.06 |
| 94 | na | na | na |
| 95 | 0.5 | 0.03 | 4.0 |
| 96 | 0.125 | 0.015 | 0.25 |
| 97 | 0.125 | 0.125 | 0.25 |
| 98 | 0.03 | 0.003 | 0.5 |
| 99 | 0.03 | 0.015 | 0.125 |
| 100 | 1.0 | 0.015 | 1.0 |
| 101 | 0.125 | 0.003 | 0.125 |
| 102 | 0.125 | 0.03 | 0.25 |
| 103 | 0.06 | 0.007 | 0.03 |
| 104 | 0.5 | 0.06 | 1.0 |
| 105 | 1.0 | 0.25 | 2.0 |
| 106 | 4.0 | 8.0 | 32 |
| 107 | 0.03 | 0.06 | 1.0 |
| 108 | 0.125 | 0.03 | 0.5 |
| 109 | 0.06 | 0.06 | 0.5 |
| 110 | 0.5 | 0.03 | 2.0 |
| 111 | 1.0 | 0.06 | 2.0 |

In the preceding table na indicates the results were not available.

Formula I compound in vivo Antibiotic Activity in a Systemic Staphylococci aureus Infection Model

Many of the compounds of Formula I (Examples 1-111, above) were evaluated for antibiotic activity in vivo, in a systemic infection model using female ICR mice. The animals were infected intraperitonially (IP) with 6.5×10⁶ CFU of an overnight culture of Staphylococcus aureus A15090 suspended in 7% mucin. The compounds of Formula I (such as Examples 1-10, above) were tested at 4 dose levels, (25, 6.25, 1.56, and 0.39 mg/kg) and were prepared in a test formulation consisting of 10% DMSO, 5% Tween 80 and 85% water. A PD₅₀ (the dose of drug given which protects 50% of mice from mortality) experiment runs for 5 days. During this time, mortality of mice was checked every day and deaths were recorded. The cumulative mortality at each dose level was used to calculate a PD₅₀ value for each compound. Surviving mice were sacrificed at the end of day 5 by CO₂ inhalation. Actual calculation of the PD₅₀ was performed with a computer program using the Spearman-Karber procedure. The solution was administered subcutaneously (SC) at 1 and 4 hours post-infection. The in-vivo efficacy, expressed as a PD₅₀ value, for compounds of Examples 1-10, when dosed SC, were found to be within the range of 0.6 to 10 mg/kg with the exception of the compound of Example 4 which had a PD50 of >10 mg/kg, The compounds of Examples 1-3 and 5-9 were also dosed intravenously (iv) one hour post infection as a single bolus of a solution in D5W (5% dextrose in water) in the same infection model. The compounds were found to have PD₅₀s within the range of 0.19 to 7.3 when iv administration was used. The compounds of examples 13, 15, 17, 19, 21-22, 24, 29-30, 33, 35, 37-38, 41, 44, 48-49, 52-56, 59-60, 62-65, 67, 69-82, 84-92, 97, 103 and 107-109 were found to have have PD₅₀s within the range of 0.3 to 10 mg/kg when administered subcutaneously as described above. The compounds of examples 11-12, 14, 16, 23, 25-26, 31-32, 39-40, 42-43, 45-47, 51, 58, 66, 83, 95-96, 102, 105 and 111 were found to have PD₅₀s >10 mg/kg. For the compounds of examples 18, 20, 27-28, 34, 50, 61, 68, 93-94, 98-101, 104, 106 and 110 no PD₅₀ data was available.

What is claimed is:

1. A compound of Formula I, including pharmaceutically acceptable salts thereof, 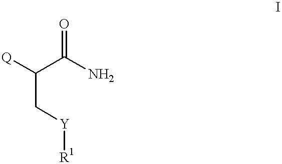
wherein:
Q is a residue of a thiazolyl peptide antibiotic selected from: [chemical structure]
Y is NR or S(O)ₘ;
m is 0, 1, or 2;
W is selected from the group consisting of hydrogen, [chemical structure]
R is selected from the group consisting of hydrogen, hydroxy, C₁₋₆alkoxy, —[(CH₂)₂O]ₚ(CH₂)₂R⁴, —C(O)C₁₋₆alkyl, —C(O)C₁₋₆alkylCO₂H, —C(O)NHC₁₋₆alkyl and C₁₋₈alkyl, in which said C₁₋₈alkyl is optionally substituted by one to six hydroxy and optionally substituted by one to two same or different substituents selected from the group consisting of (a)-(h):
(a) CO₂R⁵;
(b) SO₃H;
(c) NR⁶R⁷;
(d) heteroaryl, in which said heteroaryl is selected from the group consisting of pyridinyl, pyrimidinyl, pyrazolyl, pyrazinyl, pyrrolyl, imidazolyl, triazolyl and tetrazolyl, and in which said heteroaryl is optionally substituted with one or two same or different nitro or C₁₋₄alkyl;
(e) phenyl, in which said phenyl is optionally substituted with one to three C₁₋₄alkoxy or optionally substituted with one 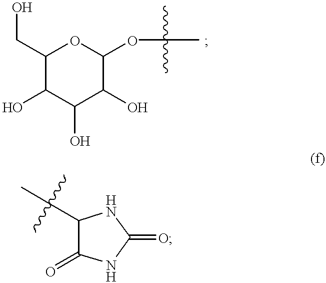
(g) C₁₋₄alkoxy; and
(h) —C(O)NH-heteroaryl, in which said heteroaryl is selected from the group consisting of pyridinyl, pyrimidinyl, pyrazolyl, pyrrolyl, imidazolyl, triazolyl and tetrazolyl;
R¹ is selected from the group consisting of: [chemical structure]
, hydrogen, —[(CH₂)₂O]_{p′}(CH₂)₂R^{4′} and C₁₋₈alkyl, in which said C₁₋₈alkyl is optionally substituted by one to six hydroxy and optionally substituted by one to two same or different substituents selected from the group consisting of (a)-(h):
(a) CO₂R^{5′};
(b) SO₃H;
(c) NR^{6′}R^{7′}
(d) heteroaryl, in which said heteroaryl is selected from the group consisting of pyridinyl, pyrimidinyl, pyrazolyl, pyrazinyl, pyrrolyl, imidazolyl, triazolyl and tetrazolyl, and in which said heteroaryl is optionally substituted with one or two same or different nitro or C₁₋₄alkyl;
(e) phenyl, in which said phenyl is optionally substituted with one to three C₁₋₄alkoxy or optionally substituted with one 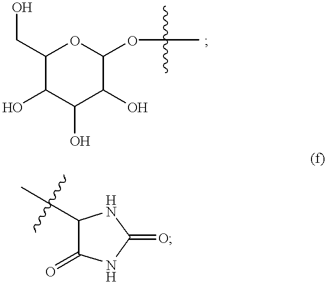
(g) C₁₋₄alkoxy; and
(h) —C(O)NH-heteroaryl, in which said heteroaryl is selected from the group consisting of pyridinyl, pyrimidinyl, pyrazolyl, pyrrolyl, imidazolyl, triazolyl and tetrazolyl;
or R and R¹ together with the nitrogen to which they are attached form a heteroalicyclic selected from the group consisting of: 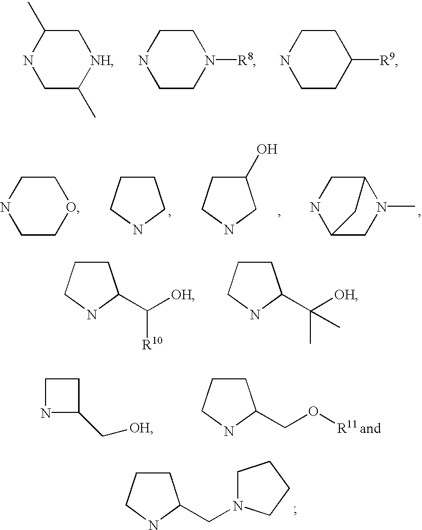
R² is selected from the group consisting of hydrogen, hydroxy, —OC(O) C₁₋₆alkyl and —OC(O)NHC₁₋₆alkyl;
R³ is hydrogen or 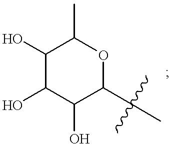
p and p′ are each independently selected from the group consisting of 1, 2 and 3;
R⁴ and R^{4′} are each independently selected from the group consisting of hydroxy, amino and C₁₋₄alkoxy;
R⁵ and R^{5′} are each independently selected from the group consisting of hydrogen, C₁₋₆alkyl and phenylmethyl;
R⁶, R^{6′}, R⁷ and R^{7′} are each independently selected from the group consisting of hydrogen, —C(O)C₁₋₆alkyl, pyridinyl and C₁₋₆alkyl, in which said C₁₋₆alkyl is optionally substituted with one hydroxy, amino, C₁₋₄alkylamino, or di(C₁₋₄alkyl)amino,
or R⁶ and R⁷ taken together with the nitrogen to which they are attached, or R^{6′} and R^{7′} taken together with the nitrogen to which they are attached form a heteroalicyclic selected from the group consisting of succinimid-1-yl, pyrrolidin-2-one-1-yl, pyrrolidin-1-yl, piperidin-1-yl, 4-hydroxypiperidin-1-yl, morpholin-4-yl, piperazin-1-yl and 4-methylpiperazin-1-yl;
R⁸ is selected from the group consisting of C₁₋₆alkyl, —C(O)C₁₋₆alkyl, —[(CH₂)₂O]_{q}(CH₂)₂R^{8′}, pyridinyl and pyrimidinyl, in which said C₁₋₆alkyl is optionally substituted with one di(C₁₋₄alkyl)amino, morpholin-4-yl, CO₂H, —CO₂C₁₋₄alkyl, tri(C₁₋₄alkoxy)phenyl and di(C₁₋₄alkoxy)pyrimidinyl;
q is 1, 2 or 3;
R^{8′} is selected from the group consisting of hydroxy, amino and C₁₋₄alkoxy;
R⁹ is hydrogen or hydroxy;
R¹⁰ is selected from the group consisting of hydrogen, C₁₋₆alkyl, C₃₋₆cycloalkyl and 1-methyl-1H-imidazol-2-yl; and
R¹¹ is C₁₋₄alkyl or pyridinyl.

2. A compound of claim 1, including pharmaceutically acceptable salts thereof, wherein:
Q is

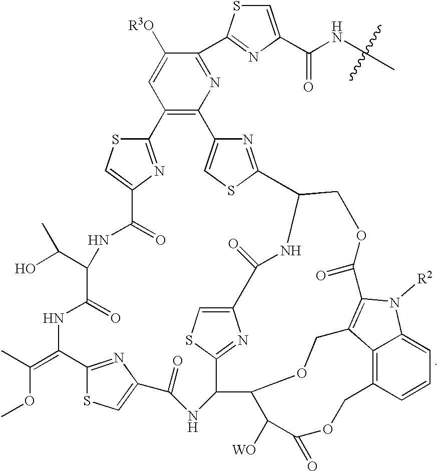

3. A compound of claim 2, including pharmaceutically acceptable salts thereof, wherein:
W is selected from the group consisting of hydrogen,

[chemical structure]

4. A compound of claim 3, including pharmaceutically acceptable salts thereof, wherein:
Y is NR.

5. A compound of claim 4, including pharmaceutically acceptable salts thereof, wherein:
R is selected from the group consisting of hydrogen, hydroxy, —C(O)CH₃, —C(O)(CH₂)₂CO₂H, —C(O)NHCH₃ and C₁₋₄alkyl, in which said C₁₋₄alkyl is optionally substituted with one hydroxy or di(C₁₋₄alkyl)amino.

6. A compound of claim 4, including pharmaceutically acceptable salts thereof, wherein:
NRR¹ is selected from the group consisting of:

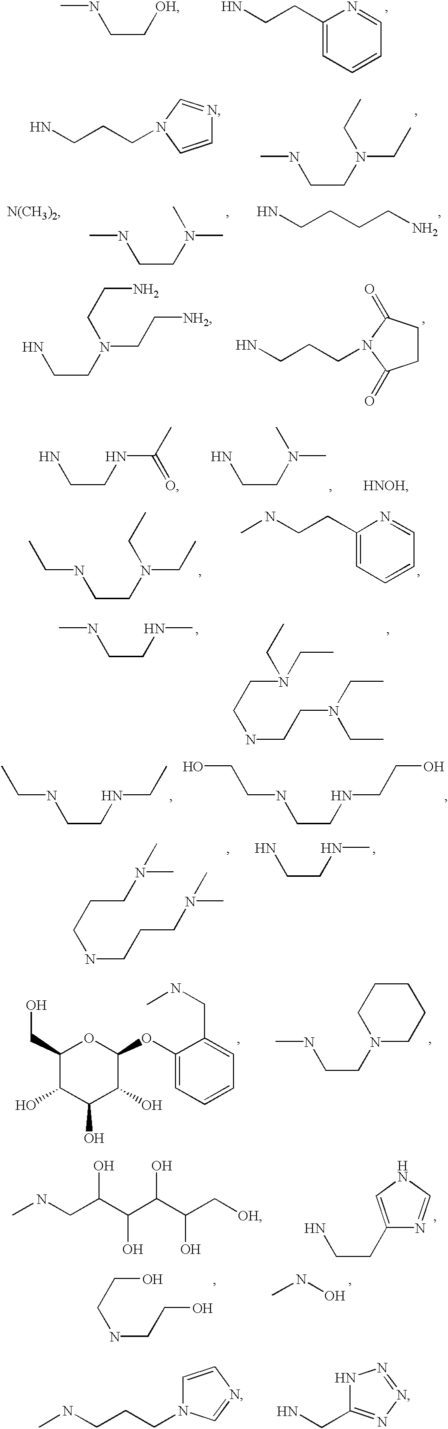

7. A compound of claim 6, including pharmaceutically acceptable salts thereof, wherein:
W is 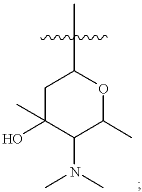
R² is hydroxy; and
R³ is hydrogen.

8. A compound of claim 6, including pharmaceutically acceptable salts thereof, wherein:
R² and R³ are each hydrogen.

9. A compound of claim 8, including pharmaceutically acceptable salts thereof, wherein:
W is

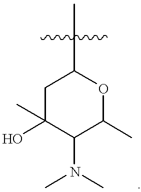

10. The compound of claim 9, including pharmaceutically acceptable salts thereof, wherein:
NRR¹ is

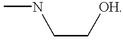

11. A compound of claim 6, including pharmaceutically acceptable salts thereof, wherein:
W is hydrogen;
R² is hydroxy; and
R³ is hydrogen.

12. A compound of claim 11, including pharmaceutically acceptable salts thereof, wherein:
NRR¹ is selected from the group consisting of:

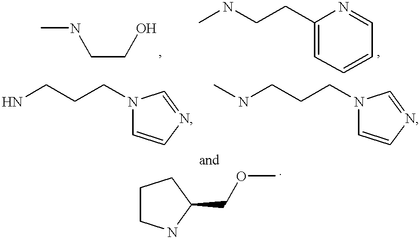

13. A compound of claim 3, including pharmaceutically acceptable salts thereof, wherein:
Y is S(O)ₘ in which m is 0 or 2;
R¹ is selected from the group consisting of CH₃, CH₂CH₃, (CH₂)₂OH, CH(CH₃)CH₂OH, CH₂[CH(OH)]₄CH₂OH, [(CH₂)₂O]₂(CH₂)₂OH, [(CH₂)₂O]₂(CH₂)₂OCH₃, [(CH₂)₂O]₂(CH₂)₂NH₂, [(CH₂)₂O]₂(CH₂)₂N(CH₃)₂, CH₂CO₂H, (CH₂)₂CO₂H, CH(CO₂H)CH₂CO₂H, CH₂CH(NHC(O)CH₃)CO₂H, (CH₂)₂SO₃H, (CH₂)₄NH₂, (CH₂)₂N(CH₃)₂, (CH₂)₃N(CH₃)₂, (CH₂)₂N(CH₂CH₃)₂, (CH₂)₂NH(CH₃), (CH₂)₂NH(CH₂CH₃), (CH₂)₂NH(CH₂)₂OH, (CH₂)₂N[(CH₂)₂NH₂)]₂, (CH₂)₂NHC(O)CH₃,

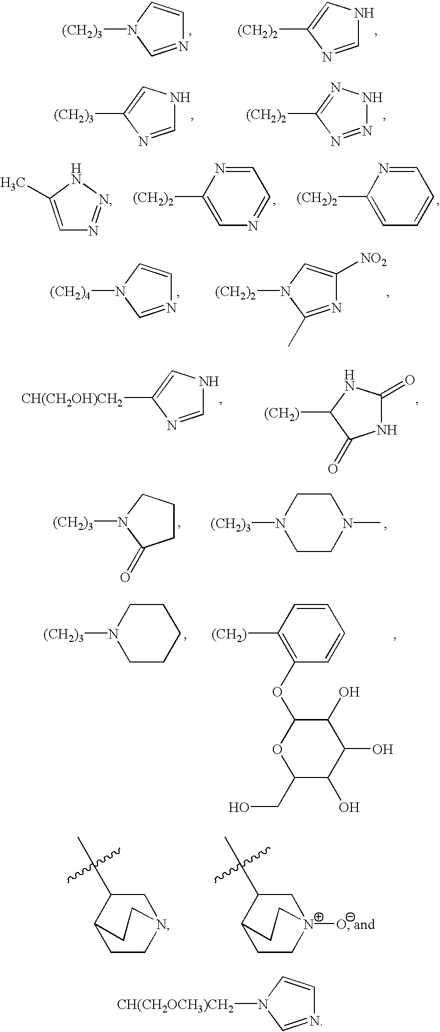

14. A compound of claim 13, including pharmaceutically acceptable salts thereof, wherein:
m is 0; and
W is hydrogen or

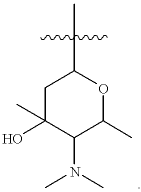

15. A compound of claim 14, including pharmaceutically acceptable salts thereof, wherein:
W is 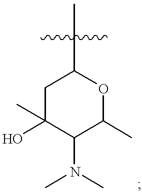
R² is hydroxy; and
R³ is hydrogen.

16. A compound of claim 15, including pharmaceutically acceptable salts thereof, wherein:
R¹ is selected from the group consisting of:
CH₂CO₂H, (CH₂)₂CO₂H, CH(CO₂H)CH₂CO₂H,
CH₂CH(NHC(O)CH₃)CO₂H, (CH₂)₂SO₃H,
(CH₂)₂N(CH₃)₂, (CH₂)₂N(CH₂CH₃)₂,

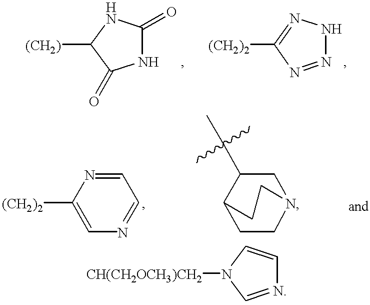

17. A compound of claim 14, including pharmaceutically acceptable salts thereof, wherein:
W is hydrogen;
R² is hydroxy; and
R³ is hydrogen.

18. A compound of claim 17, including pharmaceutically acceptable salts thereof, wherein:
R¹ is CH₂CO₂H or (CH₂)₂N(CH₂CH₃)₂.

19. A compound of claim 13, including pharmaceutically acceptable salts thereof, wherein:
m is 2; and
W is

[chemical structure]

20. A compound of claim 19, including pharmaceutically acceptable salts thereof, wherein:
R² is hydroxy; and
R³ is hydrogen.

21. A compound of claim 20, including pharmaceutically acceptable salts thereof, wherein:
R¹ is selected from the group consisting of:

[chemical structure]

22. A compound of claim 1, including pharmaceutically acceptable salts thereof, wherein:
Q is

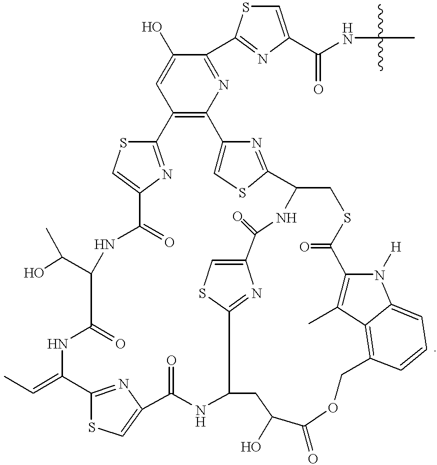

23. A compound of claim 22, including pharmaceutically acceptable salts thereof, wherein:
Y is NR.

24. The compound of claim 23, including pharmaceutically acceptable salts thereof, wherein:
R is methyl; and
R¹ is 3-(imidazol-1-yl)-propyl.

25. A compound of claim 22, including pharmaceutically acceptable salts thereof, wherein:
Y is S.

26. The compound of claim 25, including pharmaceutically acceptable salts thereof, wherein:
R¹ is (CH₂)₂N(CH₂CH₃)₂.

27. A pharmaceutical composition which comprises a therapeutically effective amount of a compound as claimed in any of claims 1-26, and a pharmaceutically acceptable carrier, adjuvant or diluent.

28. A method of treating or preventing bacterial or mycobacterial infection by administering to a mammal in need thereof a therapeutically effective amount of a compound or composition as claimed in any of claims 1-26.

29. The method of claim 28, wherein said bacterial infection is caused by a gram positive bacteria or a mycobacterium.

30. The method of claim 29, wherein said gram positive bacterial infection or mycobacterial infection is caused by methicillin-resistant Staphylococcus aureus, vancomycin-resistant Staphylococcus aureus, vancomycin-resistant Enterococcus faecalis, vancomycin-resistant Enterococcus faecium or Mycobacteria tuberculosis.
